(19) United States
(12) Patent Application Publication
(10) Pub. No.: US 2024/0307644 A1
(43) Pub. Date: Sep. 19, 2024

(54) FLEXIBLE ARTICULATING INTUBATION TOOL

(71) Applicant: TJB Medical, Inc., Columbus, MN (US)

(72) Inventors: Benn Douglas Horrisberger, Blaine, MN (US); Thomas Brian Moffat, Hugo, MN (US); David Melander, St. Louis Park, MN (US)

(21) Appl. No.: 18/268,510

(22) PCT Filed: Dec. 17, 2021
(86) PCT No.: PCT/US2021/064089

§ 371 Date: Jun. 20, 2023

Related U.S. Application Data
(60) Provisional application No. 63/155,301, filed on Mar. 1, 2021.
(60) Provisional application No. 63/127,986, filed on Dec. 18, 2020.

(51) Int. Cl. A61M 16/04; A61M 25/00; A61M 25/01

(52) U.S. Cl. CPC A61M 16/0488; A61M 25/0026; A61M 25/0074; A61M 25/0102; A61M 25/0136; A61M 25/0138; A61M 2205/586

(57) ABSTRACT

An intubation tool for assisting in placement of an endotracheal tube, the intubation tool comprising a) a stylet having a proximal end, a distal end, and a medial location, the stylet: i) configured for receiving an endotracheal tube, and ii) having an articulating distal tip, the articulating distal tip being configured to articulate between more than one shape; and b) a removable handle secured to a medial location on of the stylet.

This application is being filed as a PCT International Patent application on Dec. 17, 2021 in the name of TJB Medical, Inc., a U.S. national corporation, applicant for the designation of all countries, and Benn Douglas Horrisberger, a U.S. citizen, Thomas Brian Moffat, a U.S. citizen and David Melander, a U.S. citizen, inventors for the designation of all countries, and claims priority to U.S. Provisional Patent Application No. 63/155,301, filed Mar. 1, 2021 and U.S. Provisional Patent Application No. 63/127,986, filed Dec. 18, 2020, the contents of which are herein incorporated by reference in their entirety.

### FIELD

The present application is directed to a tool for aiding in endotracheal intubation, in particular a tool for assisting in placement of an endotracheal tube into a trachea.

### BACKGROUND

Approximately 8% of the population has partial to zero visual laryngeal exposure resulting in difficult intubation. In many instances, this partial view caused by anatomical variances inhibits placement of an endotracheal tube into the trachea. This inhibition is often due to the inability to manipulate the end of the rigid stylet and (superimposed endotracheal tube) while performing intubation. The spatial limitations imposed allow only modest movements of the stylet/endotracheal tube. Anatomical variances further inhibit the visualization of the trachea mandating precise control of the stylet/endotracheal tube for successful intubation of the trachea. Therefore, a need exists for a means to aid in placement of an endotracheal tube into a trachea.

### SUMMARY OF THE INVENTION

The present disclosure is directed to an intubation tool for assisting in placement of an endotracheal tube. The intubation tool comprises a stylet having a proximal end and a distal end, the stylet configured for receiving an endotracheal tube. The stylet has an articulating distal end, the articulating distal end being configured to articulate between more than one shape. The tool also includes a handle secured to the proximal end of the stylet.

In an embodiment, an intubation tool for assisting in placement of an endotracheal tube, the intubation tool is included having a) a stylet having a proximal end, a distal end, and a medial location, the stylet: i) configured for receiving an endotracheal tube, and ii) having an articulating distal tip, the articulating distal tip being configured to articulate between more than one shape, and b) a removable handle secured to a medial location of the stylet.

In an embodiment, articulation of the distal tip of the stylet can be accomplished with a single hand that holds the removable handle and articulates the distal end.

In an embodiment, articulation of the distal tip of the stylet can be accomplished with a single finger manipulating a steering control mounted on the stylet.

In an embodiment, articulation of the distal tip of the stylet can be accomplished with two fingers manipulating a steering control mounted on the stylet, the steering control sliding along the stylet.

In an embodiment, articulation of the distal end of the stylet can be accomplished with three fingers from a single hand that articulates the distal end.

In an embodiment, the articulating distal tip can be articulated with a force of less than 9 Newton. Generally, the force necessary move the distal tip should be relatively small so that just the simple force applied by a few figures (such as one, two, or three fingers) is adequate to manipulate the distal tip of the stylet. Note that just one, two or three fingers can be used to easily articulate the tip from a neutral position to a positive or negative position without ever releasing grip on the handle. Thus, there is a continuous range of movement from positive to neutral to distal positions (and intermediate positions), all within the fingers in contact with the tool the whole time and make the movement of the tip. This allows for very precise manipulation of the tip, but also allows for sensitive feedback to the operator's fingers. In this way the operator is able to get multiple types of feedback: They can get the feedback of resistance to articulation (and thus sensing where they are within a patient's anatomy), but also get feedback from the tip while advancing it into the patient. In this manner the operator has unparalleled awareness to the patient's anatomy and the position of the tip as it moves through the anatomy.

In an embodiment, the articulating distal tip can be articulated with a force of from 4 to 9 Newton.

In an embodiment, the articulating distal tip is deformable upon contact with an obstruction.

In an embodiment, the articulating distal tip transmits force back to steering control upon contact with an obstruction.

In an embodiment, translation movement of the steering control results in bending articulation of the articulating distal tip.

In an embodiment, the translation movement of the steering control by a distance D results in bending articulation of the distal tip by a distance of at least 150 percent of D. As used herein, D refers to the distance the steering control is moved along the axis of the stylet, either toward or away from the distal tip. For example, the distance D could be 1 centimeter, in which case 150 percent deflection of the distal tip would be 1.5 centimeters. In alternative embodiments the articulation of the distal tip can be less than 100 percent or more than 150 percent. Also, it will be appreciated that in some embodiments there is a non-linear relationship between translational movement of the steering control and bending articulation of the distal tip. Thus, the amount of translational movement can result in either increased or decreased bending (deflection) of the tip at different points along the path of travel of the steering control.

In an embodiment, translational movement of the steering control in a first direction results in bending articulation of the articulating distal tip in a first direction, and translational movement of the steering control in an opposite direction results in bending articulation of the articulating distal tip in a direction opposite the first direction.

In an embodiment, the stylet includes tubing containing at least one lumen.

In an embodiment, the stylet includes bilumen tubing.

In an embodiment, upon removal of the handle from the medial portion of the stylet, the medial portion of the stylet has a diameter no greater than 120 percent of the remainder of the stylet.

In an embodiment, the widest portion of the stylet after removal of the handle is less than the diameter of the interior of the lumen of an endotracheal tube to be installed on the stylet.

In an embodiment, the handle and stylet provide a clear sight line of angle α of 20 degrees along a dorsal elevation L_{D} of 1 cm.

In an embodiment, the handle and stylet provide a clear sight line of angle α of 20 degrees along a dorsal elevation L_{D} of 2 cm.

In an embodiment, the handle and stylet provide a clear sight line of angle α of 20 degrees along a dorsal elevation L_{D} of 3 cm.

In an embodiment, the handle and stylet provide a clear sight line of angle α of 90 degrees along a dorsal elevation L_{D} of 1 cm.

In an embodiment, the handle and stylet provide a clear sight line of angle α of 180 degrees along a dorsal elevation L_{D} of 3 cm.

In an embodiment, the handle and stylet provide a clear sight line of angle α of 270 degrees along a dorsal elevation L_{D} of 3 cm.

In an embodiment, the stylet has a length at least 1.5 times the length of an endotracheal tube to be installed on the stylet.

In an embodiment, the stylet has a length at least 2.0 times the length of an endotracheal tube to be installed on the stylet.

In an embodiment, wherein handle and stylet provide a substantially clear sight-line along the length of the stylet when the stylet is in a non-articulated mode.

In an embodiment, wherein handle and stylet provide a substantially clear sight line of at least 180 degrees along the dorsal line of the handle and stylet combination.

In an embodiment, wherein handle and stylet provide a substantially clear sight line of at least 90 degrees along a dorsal line of the handle and stylet combination, within 1 cm of the central axis of the stylet.

In an embodiment, wherein handle and stylet provide a substantially clear sight line of at least 90 degrees along a dorsal line of the handle and stylet combination, within 2 cm of the central axis of the stylet.

In an embodiment, wherein handle and stylet provide a substantially clear sight line of at least 270 degrees along a dorsal line of the handle and stylet combination, within 1 cm of the central axis of the stylet.

In an embodiment, the handle is removable from the medial location of the stylet to allow placement of an endotracheal tube.

In an embodiment, the stylet articulates in a plane.

In an embodiment, the plane in which the stylet articulates can be selected by rotation of the handle, resulting in rotation of the stylet.

In an embodiment, the stylet includes an internal mechanism for articulating the distal end.

In an embodiment, the stylet includes an internal cable connected to the distal end, and wherein pulling on the cable from the medial location results in articulating the distal end of the stylet.

In an embodiment, a method for placement of an endotracheal tube, the method is included, the method a) providing an intubation tool for assisting in placement of an endotracheal tube, the intubation tool is included, the method i) a stylet having a proximal end, distal end, and intermediate medial portion, the stylet configured for receiving an endotracheal tube, and having an articulating distal tip, the articulating distal tip being configured to articulate between more than one shape using one to three fingers, and ii) a handle secured to a medial position on the stylet, b) inserting the distal end of the stylet of the intubation tool into the trachea of a person, including articulating the distal end of the stylet during insertion to aid in passage through the larynx, c) removing the handle from the medial portion of the stylet, d) placing an endotracheal tube over the medial location of the stylet and down toward the distal end of the stylet and into the trachea of the person, and e) removing the stylet from the patient's trachea while keeping the endotracheal tube in place.

In an embodiment, the stylet articulates in a plane.

In an embodiment, the stylet includes an internal mechanism for articulating the distal tip.

In an embodiment, the stylet further includes a flexible intermediate portion.

In an embodiment, the stylet includes an internal cable connected to the distal tip, and wherein pulling on the cable from the medial location results in articulating the distal tip of the stylet.

In an embodiment, further can include an actuator.

In an embodiment, the actuator includes a tube surrounding a portion of the medial location of the stylet.

In an embodiment, wherein handle and stylet provide a substantially clear sight-line along the length of the stylet when the stylet is in a non-articulated mode.

In an embodiment, wherein handle and stylet provide a substantially clear sight line of at least 180 degrees along the dorsal line of the handle and stylet combination.

In an embodiment, wherein handle and stylet provide a substantially clear sight line of at least 90 degrees along a dorsal line of the handle and stylet combination, within 1 cm of the central axis of the stylet.

In an embodiment, wherein handle and stylet provide a substantially clear sight line of at least 90 degrees along a dorsal line of the handle and stylet combination, within 2 cm of the central axis of the stylet.

In an embodiment, wherein handle and stylet provide a substantially clear sight line of at least 270 degrees along a dorsal line of the handle and stylet combination, within 1 cm of the central axis of the stylet.

In an embodiment, an intubation tool for assisting in placement of an endotracheal tube, the endotracheal intubation tool is included having a) a stylet having a medial location and a distal end, the stylet: i.) configured for receiving an endotracheal tube, and ii) having an articulating distal tip, the articulating tip end being configured to articulate between more than one shape, and b) a handle secured to the medial location of the stylet, wherein actuation is provided by a cable.

In an embodiment, the cable radial location optimized to facilitate articulation and minimize pull force.

In an embodiment, the handle is removable from the stylet.

In an embodiment, the handle is removable from the medial location of the stylet to allow placement of an endotracheal tube.

In an embodiment, the stylet articulates in a plane.

In an embodiment, the stylet includes an internal mechanism for articulating the distal end.

In an embodiment, where the stylet further includes a flexible intermediate portion.

In an embodiment, the stylet includes an internal cable connected to the distal end, and wherein pulling on the cable from the medial location results in articulating the distal end of the stylet.

In an embodiment, further can include an actuator.

In an embodiment, the actuator includes a tube surrounding a portion of the medial location of the stylet.

In an embodiment, a method for placement of an endotracheal tube, the method is included, the method a) providing an intubation tool for assisting in placement of an endotracheal tube, the intubation tool is included, the method i) a stylet having a medial location and a distal end, the stylet configured for receiving an endotracheal tube, and having an articulating distal end, the articulating distal end being configured to articulate between more than one shape, and ii) a handle secured to the medial location of the stylet, b) inserting the distal end of the stylet of the endotracheal intubation tool into the trachea of a person, including articulating the distal end of the stylet during insertion to aid in passage through the larynx, c) removing the handle from the medial location of the stylet, d) inserting an endotracheal tube over the medial location of the stylet and down toward the distal end of the stylet, and e) removing the stylet from the patient's trachea while keeping the endotracheal tube in place, wherein the actuation can be performed with a single hand.

In an embodiment, the endotracheal intubation tool for assisting in placement of an endotracheal tube is removable from the medial location of the stylet.

In an embodiment, wherein actuation occurs push/pull action on the tip by allowing 3 finger, 360 degree contact of circular trigger.

In an embodiment, the endotracheal intubation stylet includes an internal mechanism for articulating the distal end.

In an embodiment, the stylet further includes a flexible intermediate portion.

In an embodiment, the stylet includes an internal cable connected to the distal end, and wherein pulling on the cable from the medial location results in articulating the distal end of the stylet.

In an embodiment, further can include an actuator.

In an embodiment, the actuator includes a tube surrounding a portion of the medial location of the stylet.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following drawings, in which:

FIG. 1 is a drawing showing a side plan view of endotracheal intubation tool for assisting in placement of an endotracheal tube made in accordance with an example embodiment.

FIG. 2 is a drawing showing the position of a patient's head and neck in advance of insertion of an endotracheal tube, showing the approximate oral axis, pharyngeal axis and laryngeal axis.

FIG. 3 is a drawing showing a patient with an endotracheal intubation tool for assisting in placement of an endotracheal tube during initial insertion of the distal end of the endotracheal intubation tool through the patient's mouth.

FIG. 4 is a drawing showing a patient with an endotracheal intubation tool for assisting in placement of an endotracheal tube during initial insertion of the distal end of the endotracheal intubation tool through the patient's mouth, the tool substantially inserted into the patient.

FIG. 5 is a drawing showing a patient with an endotracheal intubation tool for assisting in placement of an endotracheal tube during initial insertion of the distal end of the endotracheal intubation tool through the patient's mouth, the tool substantially inserted into the patient and with the handle removed.

FIG. 6 is a drawing showing a patient with a endotracheal intubation tool for assisting in placement of an endotracheal tube during initial insertion of the distal end of the endotracheal intubation tool through the patient's mouth, the tool substantially inserted into the patient and with the handle removed and the endotracheal tube positioned over the tool stylet and into the patient.

FIG. 7 is a drawing showing a side plan view of endotracheal intubation tool for assisting in placement of an endotracheal tube made in accordance with an example embodiment, showing the stylet of the tool with a removable handle clipped in place.

FIG. 8 is a drawing showing a side plan view of endotracheal intubation tool for assisting in placement of an endotracheal tube made in accordance with an example embodiment, showing the stylet of the tool with the handle removed.

FIG. 9 is a front view of the distal side of a handle of an endotracheal intubation tool with the stylet of the tool installed.

FIG. 10 is a front view of the distal side of a handle of an endotracheal intubation tool with the stylet of the tool installed and handle catch open.

FIG. 11 is a front view of the distal side of a handle of an intubation tool with the stylet of the tool removed and handle catch open.

FIG. 12 is a diagram showing the field of view from an intubation tool made in accordance with the present disclosure.

FIG. 13 is a diagram showing the field of view from an endotracheal intubation tool made in accordance with the present disclosure.

FIG. 14 is view of the distal end of an intubation tool stylet, showing the tip of the stylet in a neutral position.

FIG. 15 is view of the distal end of an intubation tool stylet, showing the tip of the stylet in a downwardly flexed position.

FIG. 16 is view of the distal end of an intubation tool stylet, showing the tip of the stylet in an upwardly flexed position.

FIG. 17 is a closeup view of the steering base of an intubation tool stylet.

FIG. 18 is view of the distal end of an intubation tool stylet, with the tip of the stylet articulated

FIG. 19 is close up view of the medial portion of a stylet, showing the steering control in a substantially neutral location.

FIG. 20 is close up view of the medial portion of a stylet, showing the steering control in a substantially distal location.

FIG. 21 is close up view of the medial portion of a stylet, showing the steering control in a substantially proximal location.

FIG. 22 is a drawing showing an exploded view of components of an endotracheal intubation tool for assisting in placement of an endotracheal tube made in accordance with an example embodiment.

FIG. 23 is a perspective view of a steering base made in accordance with an example embodiment.

FIG. 24 is a side elevational view of a steering base made in accordance with an example embodiment.

FIG. 25 a distal end view of a steering base made in accordance with an example embodiment.

FIG. 26 is a cross sectional view of a steering base made in accordance with an example embodiment.

FIG. 27 is a perspective view of the proximal end cap of an intubation tool stylet in accordance with an embodiment.

FIG. 28 is a distal end view of the proximal end cap of an intubation tool stylet in accordance with an embodiment.

FIG. 29 is a side view of the proximal end cap of an intubation tool stylet in accordance with an embodiment.

FIG. 30 is a perspective view of steering cable tip of an intubation tool stylet in accordance with an embodiment.

FIG. 31 is a proximal end view of steering cable tip of an intubation tool stylet in accordance with an embodiment.

FIG. 32 is a side view of steering cable tip of an intubation tool stylet in accordance with an embodiment.

FIG. 33 is a perspective view of steering control of an intubation tool stylet in accordance with an embodiment.

FIG. 34 is an end view of steering control of an intubation tool stylet in accordance with an embodiment.

FIG. 35 is a side view of steering control of an intubation tool stylet in accordance with an embodiment, with a partial cross section.

FIG. 36 is a perspective view of an articulation collar of an intubation tool stylet in accordance with an embodiment.

FIG. 37 is a side view of an articulation collar of an intubation tool stylet in accordance with an embodiment.

FIG. 38 is an end view of an articulation collar of an intubation tool stylet in accordance with an embodiment.

FIG. 39 is a drawing showing a side plan view of endotracheal intubation tool for assisting in placement of an endotracheal tube made in accordance with an example embodiment.

FIG. 40 is closeup of the endotracheal intubation tool of FIG. 39, enlarging the portion from dotted circle 39A.

FIG. 41 is a drawing showing an exploded view of components of an endotracheal intubation tool for assisting in placement of an endotracheal tube made in accordance with an example embodiment.

FIG. 42 is a drawing showing a side perspective view of endotracheal intubation tool for assisting in placement of an endotracheal tube made in accordance with an example embodiment, showing the articulating tip in an upward (or dorsal) articulation.

FIG. 43 is a drawing showing a side perspective view of endotracheal intubation tool for assisting in placement of an endotracheal tube made in accordance with an example embodiment, showing the articulating tip in a downward (or ventral) articulation.

FIG. 44 is drawing showing the articulating tip of an endotracheal intubation tool in a neutral position.

FIG. 45 is a perspective view of steering control of an intubation tool stylet in accordance with an embodiment.

FIG. 46 is a side view of steering control of an intubation tool stylet in accordance with an embodiment, with a partial cross section.

FIG. 47 is an end view of steering control of an intubation tool stylet in accordance with an embodiment.

FIG. 48 is a perspective view of a vertebrate from the articulating end of an articulating tip, showing the vertebrate independent of other components.

FIG. 49 is a side view of a vertebrate from the articulating end of an articulating tip, showing the vertebrate independent of other components.

FIG. 50 is an end view of a vertebrate from the articulating end of an articulating tip, showing the vertebrate independent of other components.

FIG. 51 is a perspective view of the tip member from an articulating end of an articulating tip, showing the tip member independent of other components.

FIG. 52 is a side cross-sectional view of the tip member from an articulating end of an articulating tip, showing the tip member independent of other components.

FIG. 53 is an end view of the tip member from an articulating end of an articulating tip, showing the tip member independent of other components.

FIG. 54 is a perspective view of a base member from the proximal end of an articulating tip, showing the base member independent of other components.

FIG. 55 is a side view of a base member from the proximal end of an articulating tip, showing the base member independent of other components.

FIG. 56 is a side elevational view of components of an articulating tip of an endotracheal intubation tool in a neutral position.

FIG. 57 is a side elevational view of components of an articulating tip of an endotracheal intubation tool in a partially articulated position.

FIG. 58 is a side elevational view of components of an articulating tip of an endotracheal intubation tool in a partially articulated position.

FIG. 59 is a side elevational view of components of an articulating tip of an endotracheal intubation tool in a partially articulated position.

FIG. 60 is a side elevational view of components of an articulating tip of an endotracheal intubation tool in a partially articulated position.

FIG. 61 is a side elevational view of components of an articulating tip of an endotracheal intubation tool in a partially articulated position.

FIG. 62 is a side elevational view of components of an articulating tip of an endotracheal intubation tool in a partially articulated position.

FIG. 63 is a side elevational view of components of an articulating tip of an endotracheal intubation tool in a partially articulated position, making contact with an obstruction.

FIG. 64 is a side elevational view of components of an articulating tip of an endotracheal intubation tool in a partially articulated position, making contact with an obstruction.

FIG. 65 is a side view of a handle of an endotracheal intubation tool with the stylet of the tool removed.

FIG. 66 is a perspective view of a clasp of a handle of an endotracheal intubation tool.

FIG. 67 is a perspective view of a clasp of a base of an endotracheal intubation tool.

FIG. 68 is a graph showing force relative to articulation position of an articulating tip of an endotracheal intubation tool.

FIG. 69 is a graph showing force relative to articulation position of an articulating tip of an endotracheal intubation tool.

FIG. 70 is a side cross-sectional of a vertebrate from an articulating end of an articulating tip, showing the vertebrate independent of other components.

FIG. 71 is a graph showing required to articulate a vertebrate relative to flat portion of the vertebrate.

While embodiments are susceptible to various modifications and alternative forms, specifics thereof have been shown by way of example and drawings and will be described in detail. It should be understood, however, that the scope herein is not limited to the particular embodiments described. On the contrary, the intention is to cover modifications, equivalents, and alternatives falling within the spirit and scope herein.

### DETAILED DESCRIPTION

The present disclosure is directed to a device for aiding in endotracheal intubation, in particular a tool for assisting in placement of an endotracheal tube into a trachea.

The device (also referred to herein as a “tool”) includes a) a stylet having a proximal end, a distal end, and a medial location, the stylet: i) configured for receiving an endotracheal tube, and ii) having an articulating distal tip, the articulating distal tip being configured to articulate between more than one shape, and b) a removable handle secured to a medial location on of the stylet. In an embodiment the intubation tool comprises a stylet having a proximal end and a distal end, the stylet configured for receiving an endotracheal tube. The stylet has an articulating distal end, the articulating distal end being configured to articulate between more than one shape. The tool also includes a handle secured to the proximal end of the stylet. Optionally articulation of the distal tip of the stylet can be accomplished with a single hand that holds the removable handle and articulates the distal end; and optionally articulation of the distal tip of the stylet can be accomplished with a single finger or two fingers manipulating a steering control mounted on the stylet.

The device provides a means for a clinician to apply a low axial force to the articulation collar in order to fully articulate the distal end. The means by which this is possible comes from having a low friction mechanism that allows the cable to slidably pass around the proximal transition of the steering base. This mechanism/design can include a pully-type mechanism or a design which optimizes radius and surface finish to provide a low friction surface and pathway. This radius and surface finish chosen will be highly dependent on the materials and construction of the cable itself. In order to provide tactile feedback to the clinician, who is manipulating the medial section of the device, the distal tip typically is in intimate contact with the next most proximal, rigid component, and that component to the next and so on. This contact can be accomplished by pre-tensioning the articulation cable, which places the device in tension from the proximal steering transition to the distal tip of the device. For the benefits of all of these design features to be realized, they must all fit within a narrow profile, such as a 6 mm diameter envelope so as to facilitate the sliding of a standard intubation tube along its length.

The device allows for two-way articulation in a plane. This two-way can be quite important because it allows more precise placement of the stylet of the device while navigating the tip of the stylet through a patient's anatomy. Specifically, for example, a clinician must typically cause the tip of the stylus to first pass under the epiglottis, and then flex steeply upward to move past it, next straighten to advance deeper, then flex downward steeply to get over the subglottis, past the vocal chords, into the trachea. A single directional bend is not sufficient to manage entrance in a difficult airway without disturbing a patient's tissue, often causing discomfort and even potential injury.

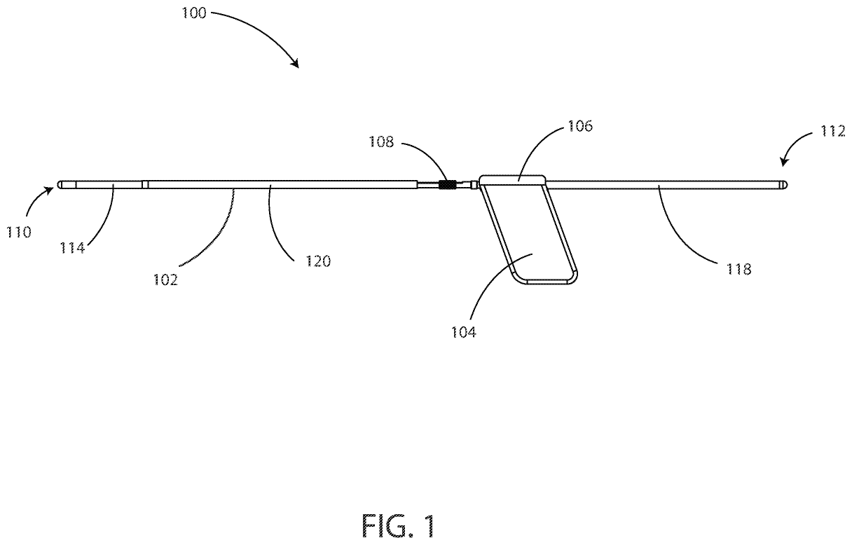

Now in reference to the drawings, FIG. 1 is a drawing showing a side plan view of intubation tool 100 for assisting in placement of an endotracheal tube made in accordance with an example embodiment. The intubation tool 100 includes a stylet 102 having distal end 110 and proximal end 112 that is flexible and has an articulating tip 114 that allows for precise placement during intubation of a patient, thereby allowing placement with minimal disturbance or damage to the patient. The intubation tool 100 further includes a removable handle 104, shown in an example configuration (with other configurations possible). The removable handle 104 can be gripped during an intubation procedure during which the stylus is placed down the trachea of a patient. After the stylus is positioned within the patient the removable handle 104 is removed and an intubation tube slipped over the stylet 102 and into the patient's trachea (the stylet 102 serving as a guide for the intubation tube), after which the stylet 102 is removed while the intubation tube remains extended partially into the trachea of the intubated patient. Removal of the handle 104 is accomplished in the depicted embodiment by opening clasp 106, as shown later herein. The stylet 102 includes a steering control 108 that can slide forward and backward so as to change the shape of articulating tip 114. Stylet 102 further includes a proximal portion 118 and a distal portion 120, both of which are typically flexible but not articulating. Proximal portion 118 of stylet 102 remains outside of a patient during the intubation procedure, while at least some of distal portion 120 is inserted into the patient.

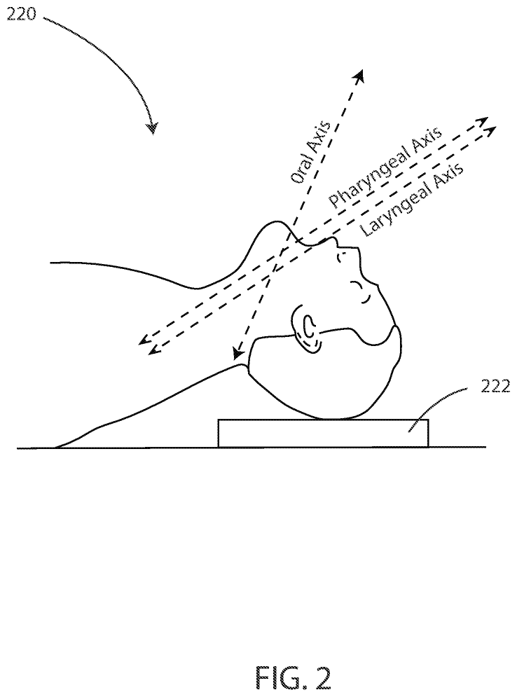

FIG. 2 is a drawing showing the position of the head and neck of a patient 220 in advance of insertion of an endotracheal tube, showing the approximate oral axis, pharyngeal axis and laryngeal axis. The patient's head is shown elevated and tilted by support 222 so as to open up access to the patient's airway. As shown in FIG. 2 the oral axis crosses the pharyngeal axis and laryngeal axis, but is not perfectly in-line, and can change as the head of the patient tilts. This geometry can vary by patient and results in challenges during intubation because the pathway for inserting the intubation tube is often partially obscured and/or can be somewhat curved or non-linear.

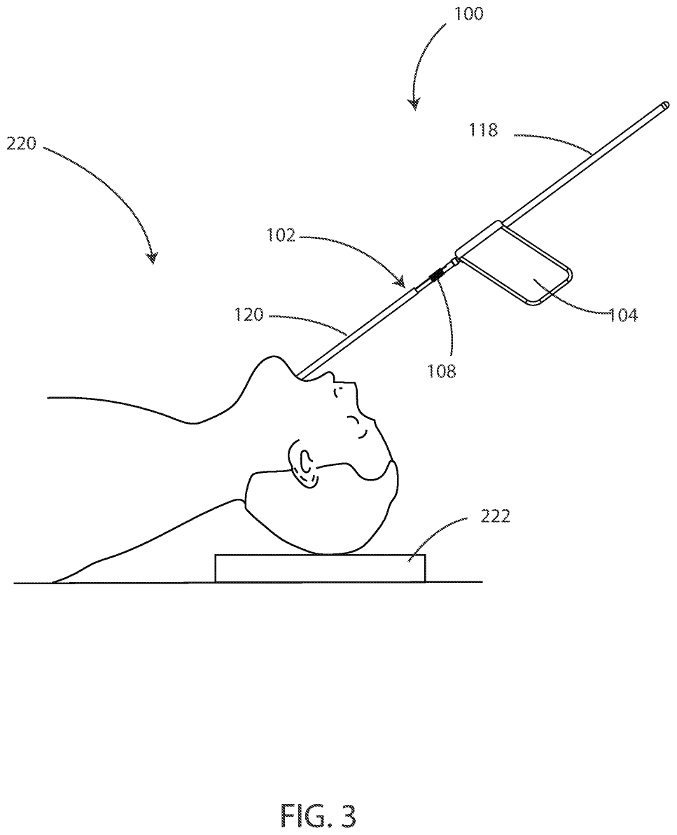

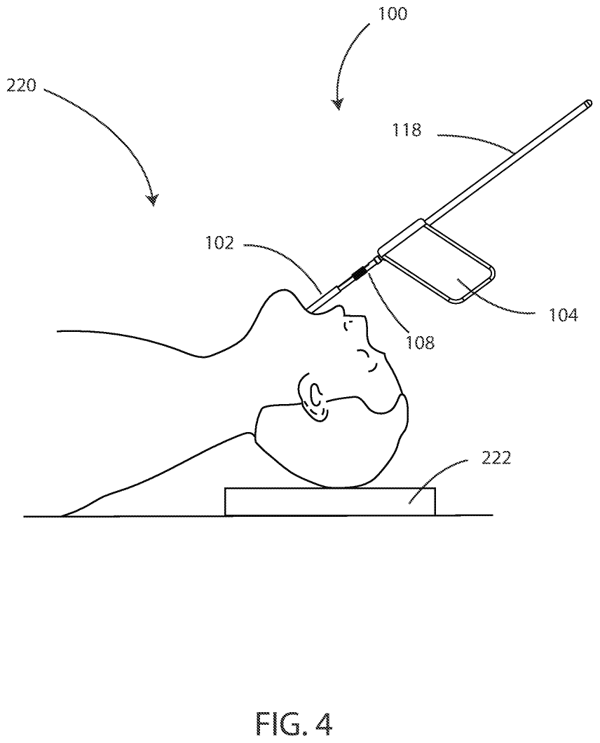

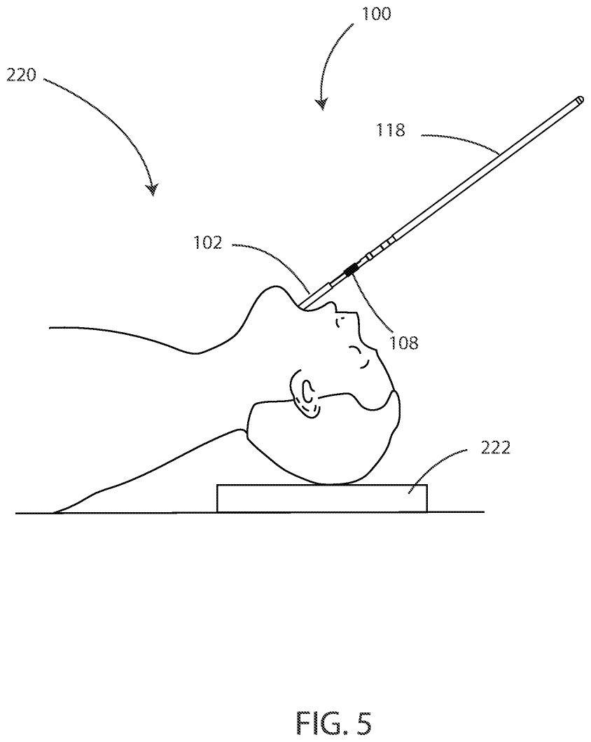

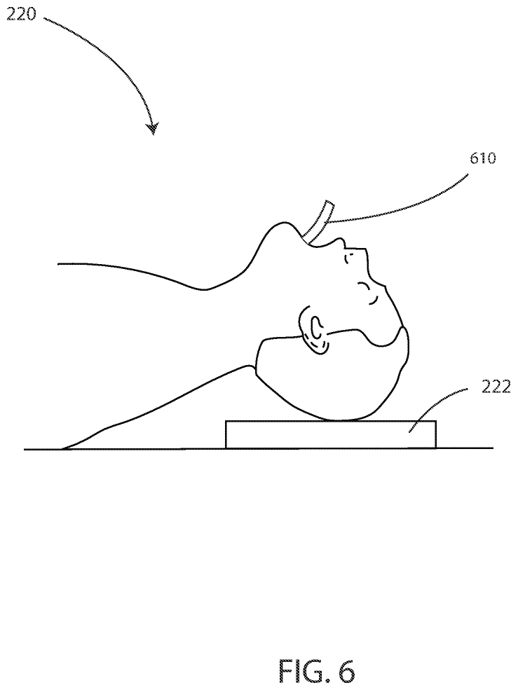

FIG. 3 is a drawing showing a patient an intubation tool 100 during initial insertion of the distal end of stylet 102 inserted through the mouth of the patient. FIG. 4 shows the stylet 102 extended further into the patient; while FIG. 5 shows the stylet 102 within a patient and the handle removed so that the stylet 102 remains, with proximal portion 118 extending out of the patient's mouth and ready for placement of the intubation tube (not shown). FIG. 6 is a drawing showing a patient with an endotracheal tube 610 emplaced.

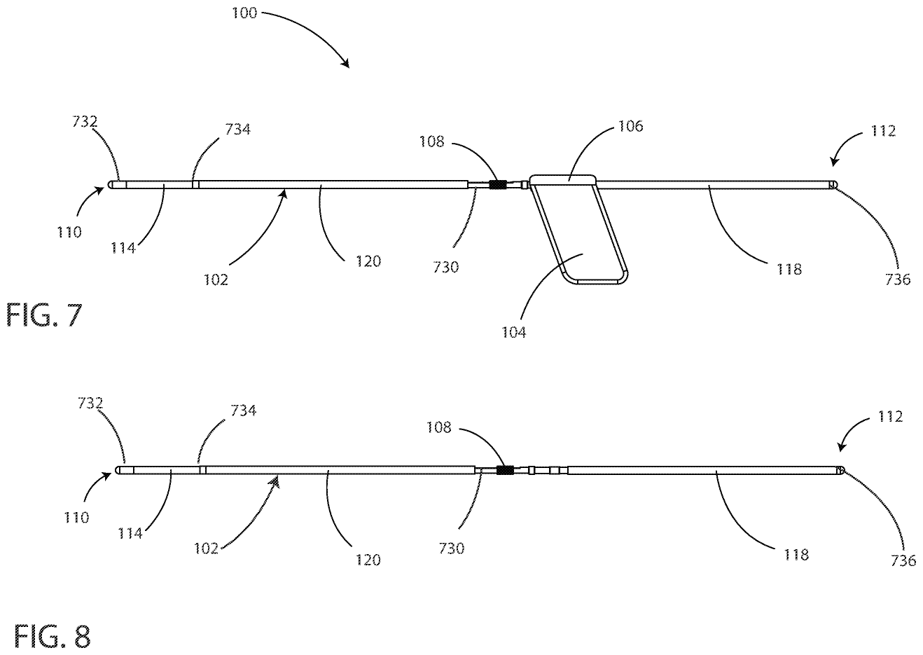

FIG. 7 is a drawing showing a side plan view of the endotracheal intubation tool 100 for assisting in placement of an endotracheal tube made in accordance with an example embodiment, showing the stylet of the tool with a removable handle 104 clipped in place. FIG. 8 is a drawing showing a side plan view of endotracheal intubation tool 100 the handle removed. The intubation tool 100 includes a stylet 102 having distal end 110 and proximal end 112 that is flexible and has an articulating tip 114 that allows for precise placement during intubation of a patient, thereby allowing placement with minimal disturbance or damage to the patient. The stylet 102 further includes a steering base 730 onto which a steering control 108 is mounted. The steering control 108 is joined to a cable (not shown) that travels down distal portion 120 of the stylet, through articulation collar 734 and to steering cable tip 732, where the ends of the steering cable are secured. By moving the steering control 108 forward and backward (using as little as one, two, or three fingers (for example)) the articulating tip is articulated (or bent) by forces delivered by the cable connecting the steering control 108 to steering cable tip 732. Stylet 102 further includes a tube end cap 736 on the proximal end of the stylet 102. It is this tube end cap 736 over which the distal end of an intubation tube passes when the intubation tube is passed over the stylet during intubation.

Removal of the handle 104 is accomplished in the depicted embodiment by opening clasp 106, as shown later herein. The stylet 102 includes a steering control 108 that can slide forward and backward so as to change the shape of articulating tip 114. Stylet 102 further includes a proximal portion 118 and a distal portion 120, both of which are typically flexible but not articulating. Proximal portion 118 of stylet 102 remains outside of a patient during the intubation procedure, while at least some of distal portion 120 is inserted into the patient.

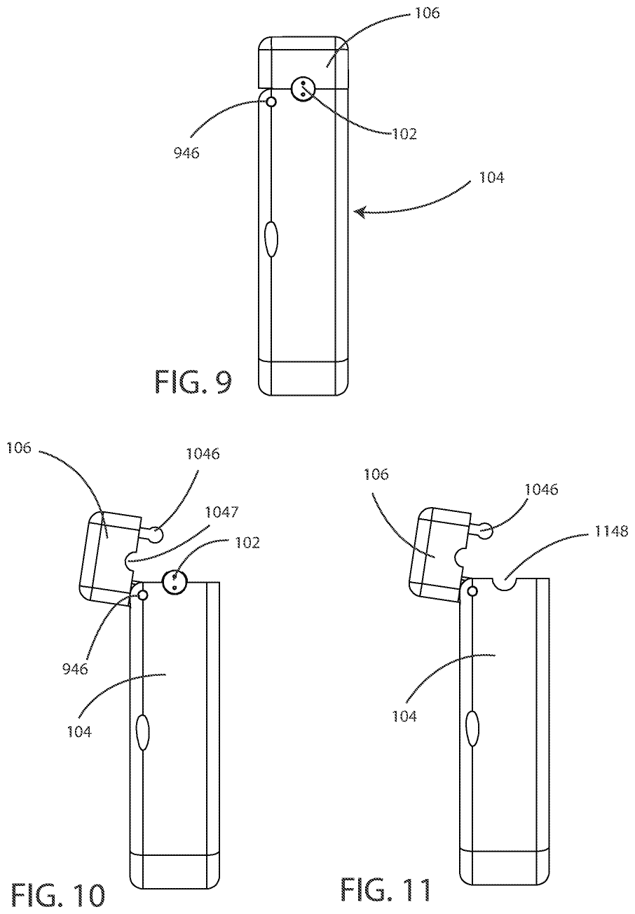

FIG. 9 is a front view of the distal side of a handle 104 of an intubation tool with the stylet 102 of the tool installed; while FIG. 10 is a front view of the distal side of the handle 104 of the intubation tool with the clasp 106 open, and FIG. 11 is a front view of the distal side of the handle 104 of the intubation tool with the tool removed and handle catch open. FIG. 9 shows a dowel 946 (typically made of metal, such as stainless steel) that the clasp 106 pivots around. In addition, the clasp 106 shows a locking member 1046 for snapping into the lower portion of the handle 104. The clasp 106 further includes a recess 1047, while the bottom of the handle 104 includes a recess 1148. Recess 1047 and 1148 combine to surround and hold the stylet 102 when the handle 104 is retained on the stylet 102.

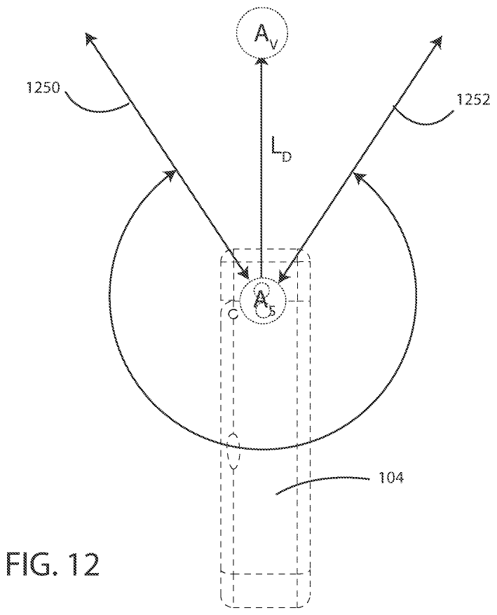

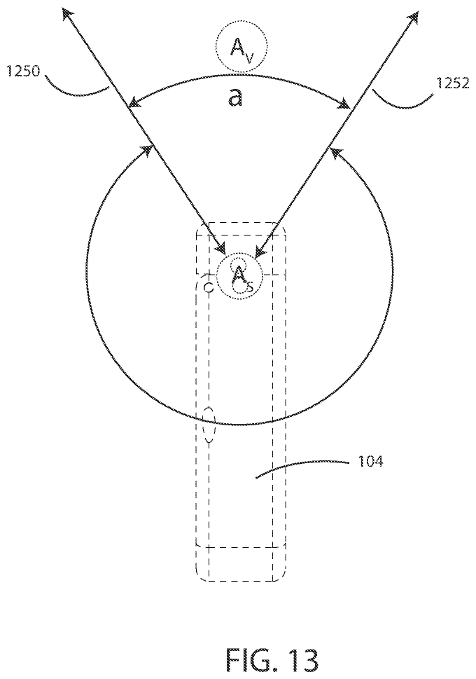

FIG. 12 is a diagram showing the field of view from intubation tool made in accordance with the present disclosure. FIG. 13 is a diagram showing the field of view from intubation tool made in accordance with the present disclosure. Both figures show how the intubation tool is designed to allow for improved visibility into a patient during medical procedures. Generally, a relatively unobstructed view is possible relative to alternative devices. In an embodiment, the handle and stylet provide a substantially clear sight line of at least 180 degrees along the dorsal line of the handle and stylet combination. In an embodiment, the handle and stylet provide a substantially clear sight line of at least 90 degrees along a dorsal line of the handle and stylet combination, within 1 cm of the central axis of the stylet. In an embodiment, the handle and stylet provide a substantially clear sight line of at least 90 degrees along a dorsal line of the handle and stylet combination, within 2 cm of the central axis of the stylet. In an embodiment, the handle and stylet provide a substantially clear sight line of at least 270 degrees along a dorsal line of the handle and stylet combination, within 1 cm of the central axis of the stylet.

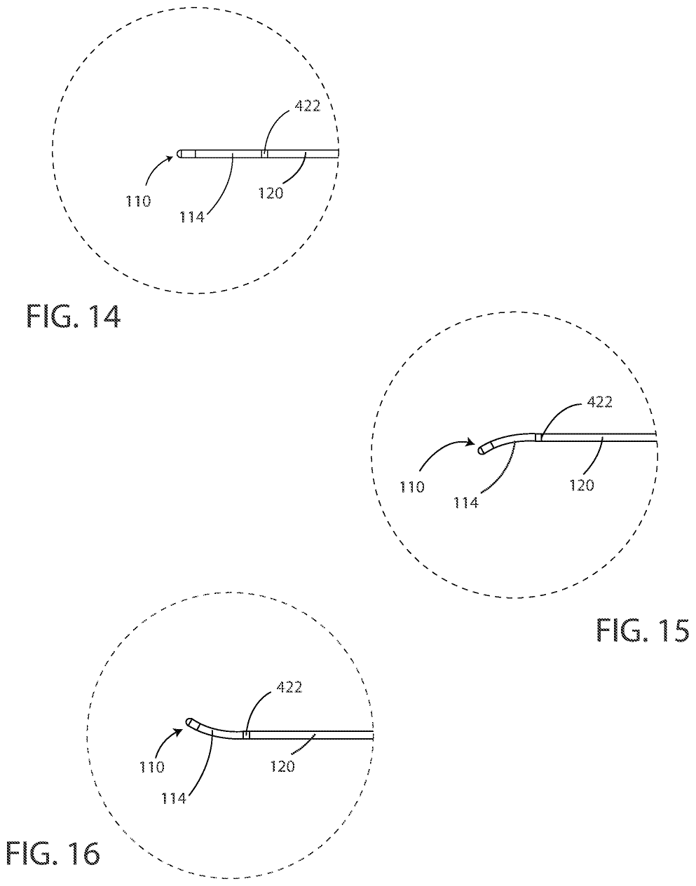

FIG. 14 is view of the distal end of an intubation tool stylet, showing the articulating tip 114 of the stylet 102 in a neutral position; FIG. 15 is view of the distal end of the stylet, showing articulating tip 114 in an downwardly flexed position; FIG. 16 is view of the distal end of stylet 102, showing the tip of the stylet in an upwardly flexed position. In this neutral position the distal end allows for insertion into a patient without trauma to the patient's tissue.

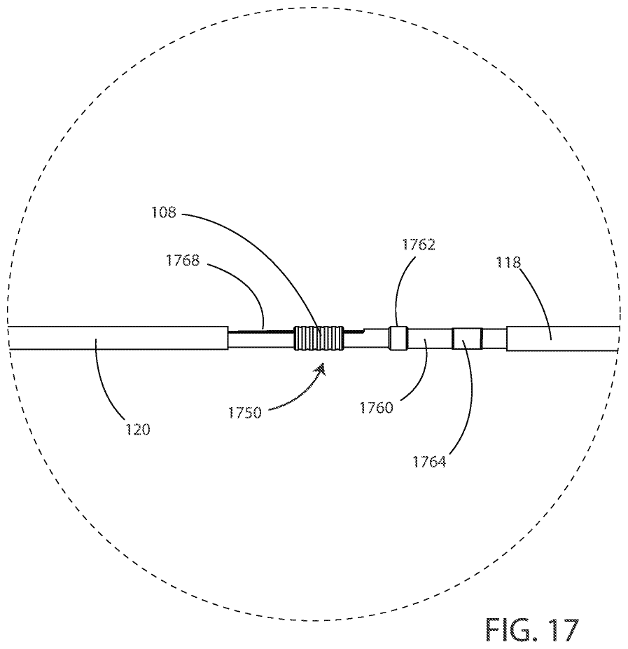

FIG. 17 is a closeup view of the steering base of an intubation tool stylet 102. The steering base 1750 includes steering control 108 that readily moves to a distal (left) location or proximal (right) location with the force of one or more fingers. The steering base 1750 includes a path 1768 on which the steering control 108 travels. This path 1768 is generally exposed even when the stylet 102 is mounted on a handle (not shown), allowing the operator to move the steering control 108 so as to articulate the tip of the stylet 102. Further, a proximal section 1760 of the steering base 1750 is shown, this proximal section 1760 including (in this embodiment) regions 1762 and 1764 that are wider areas of the steering base 1750 deigned to secure the stylet 102 in a handle and prevent rotation and sliding of the stylet.

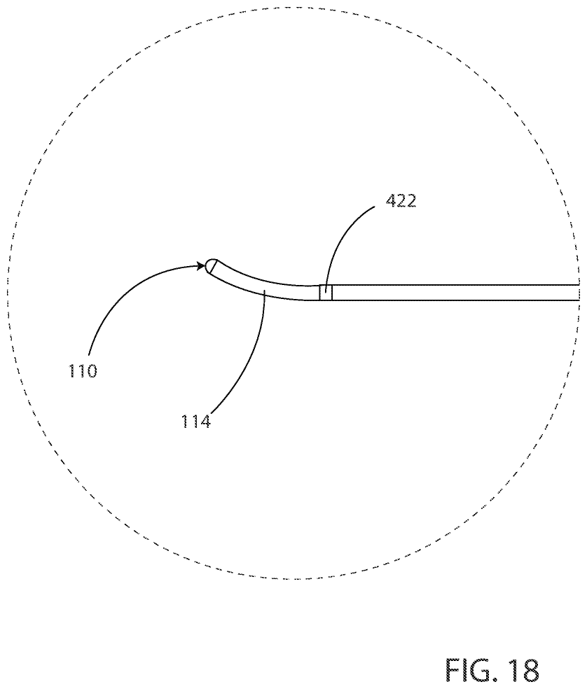

FIG. 18 is view of the distal end of an intubation tool stylet, with the tip of the stylet articulated.

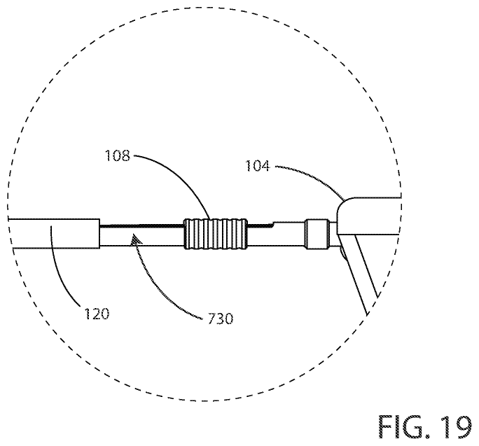

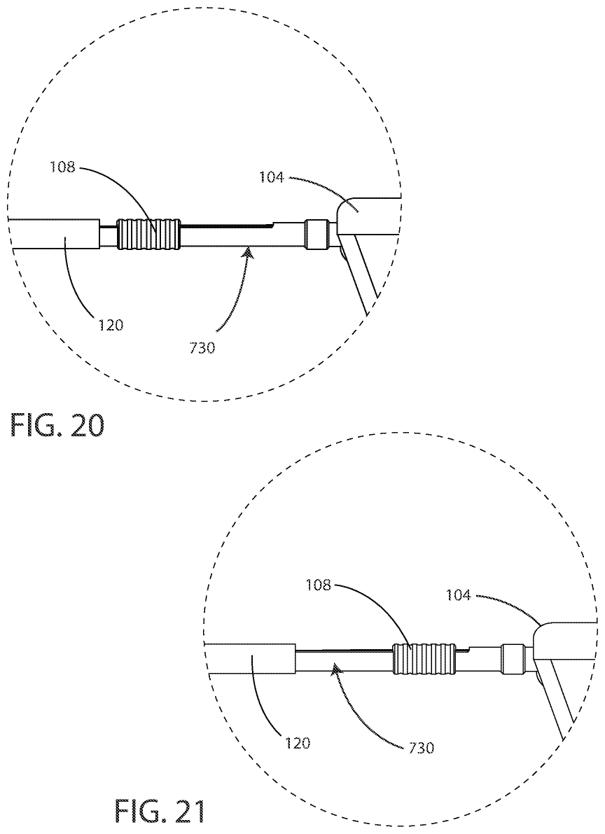

FIG. 19 is close up view of the medial portion of a stylet 102, showing the steering control 108 in a substantially neutral location; FIG. 20 is close up view of the medial portion of a stylet 102, showing the steering control 108 in a substantially distal location; FIG. 21 is close up view of the medial portion of a stylet 102, showing the steering control 108 in a substantially proximal location.

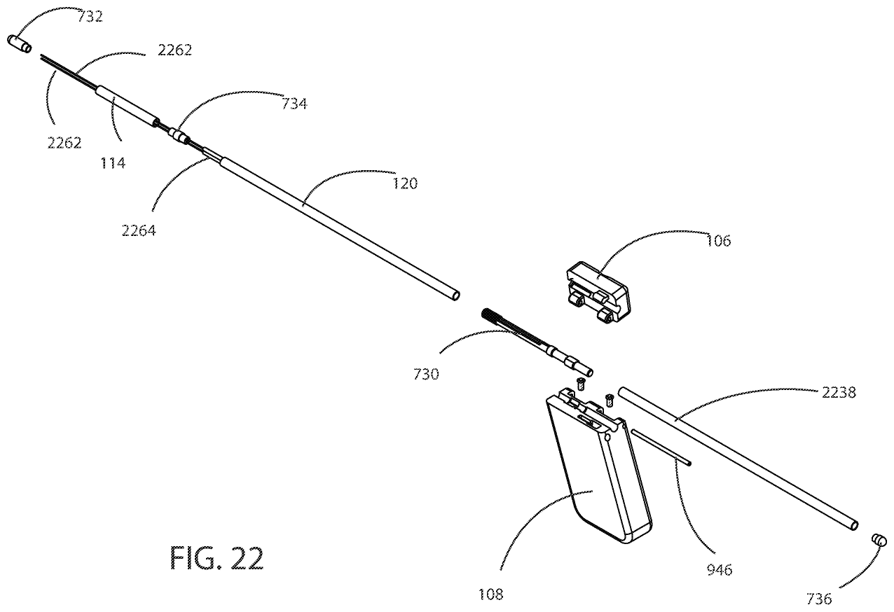

FIG. 22 is a drawing showing an exploded view of components of an intubation tool for assisting in placement of an endotracheal tube made in accordance with an example embodiment; further showing two ends of steering cable 2262 and also optional bowden cable 2264 through which the steering cable 2262 passes. The steering cable 2262 terminates in the steering cable tip 732, while also being connected to steering control 108.

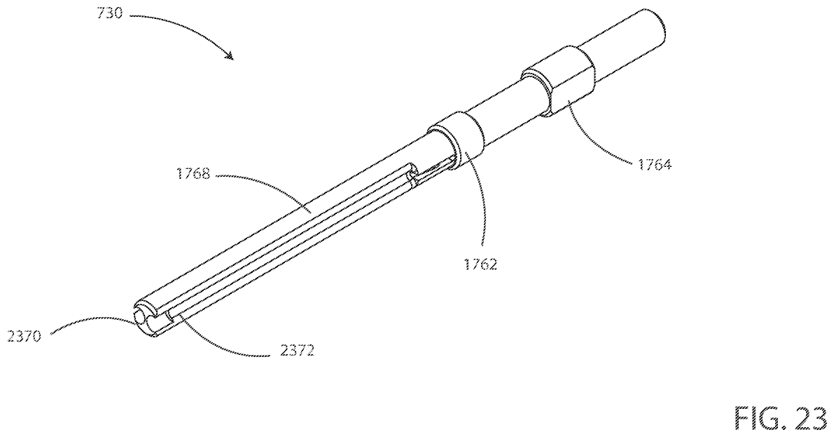

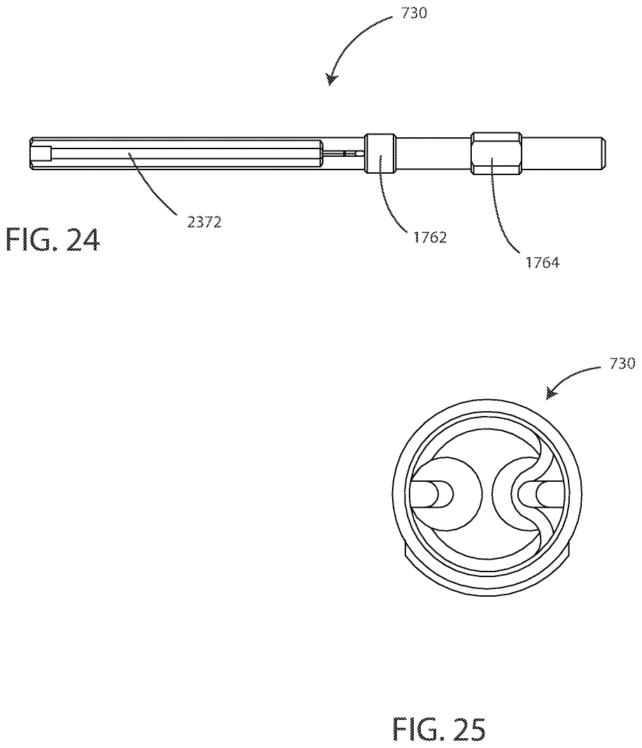

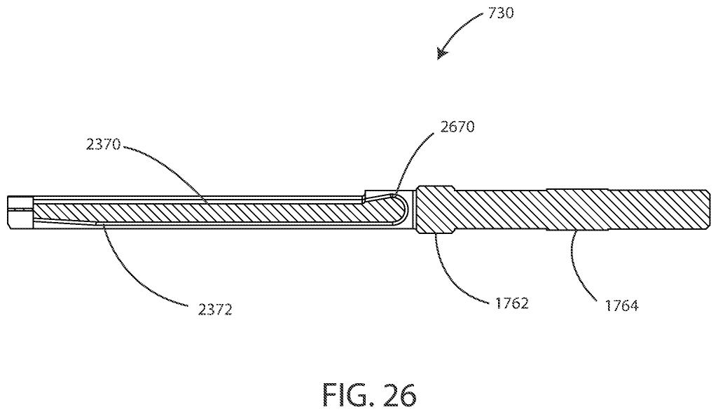

FIG. 23 is a perspective view of a steering base 730 made in accordance with an example embodiment; FIG. 24 is a side elevational view of the steering base 730; FIG. 25 a distal end view of a steering base 730; and FIG. 26 is a cross sectional view of the steering base 730. Channels 2370 and 2372 are shown in steering base 730, and these channels provide pathway 2670 for the steering cable 2262. Sleeve 2238 on the proximal end the stylet is further shown.

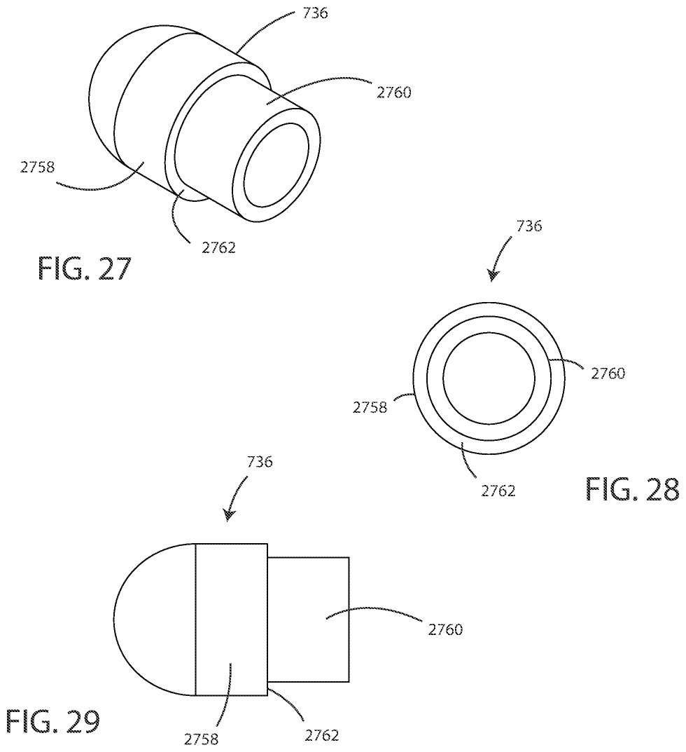

FIG. 27 is a perspective view of the proximal end cap 736 of an intubation tool stylet in accordance with an embodiment; FIG. 28 is a distal end view of the proximal end cap 736; and FIG. 29 is a side view of the proximal end cap. Exposed portion 2758 is shown, along with recessed portion 2760 for insertion into the proximal end of the stylet, along with lip 2762 to limit insertion depth of the proximal end cap.

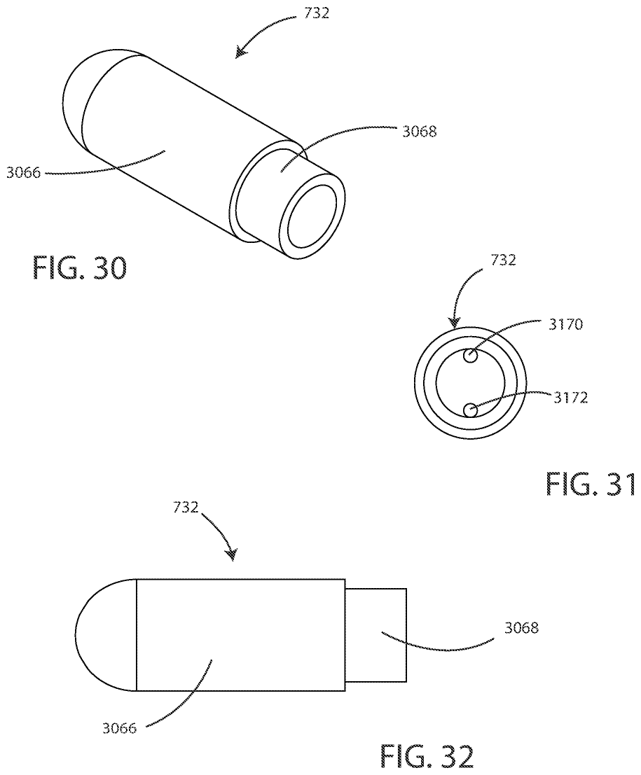

FIG. 30 is a perspective view of steering cable tip 732 of an intubation tool stylet in accordance with an embodiment; FIG. 31 is a proximal end view of steering cable tip; and FIG. 32 is a side view. Exposed portion 3066 is shown, along with recessed portion 3068 as are cable retaining holes 3170 and 3172 are shown.

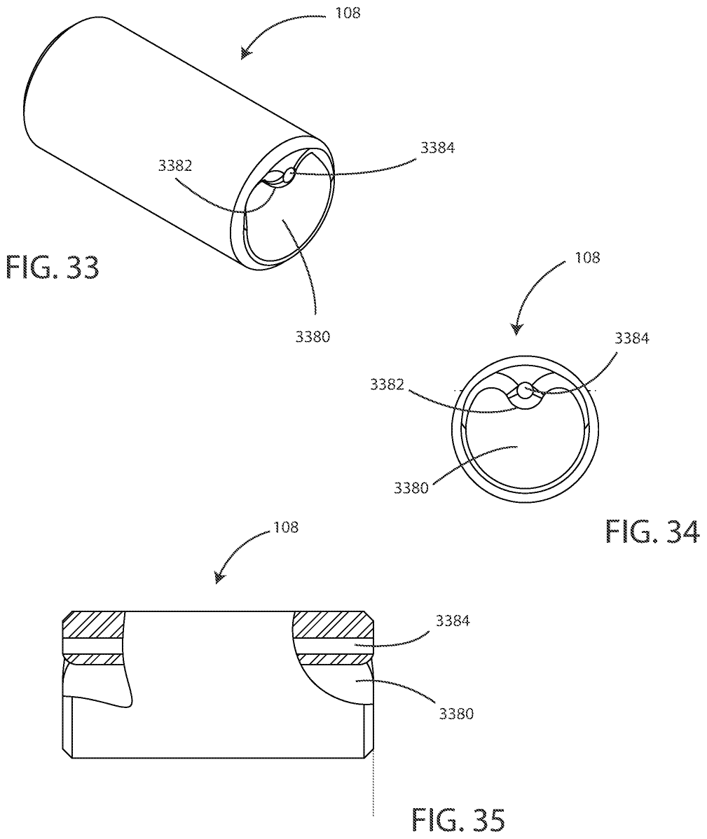

FIG. 33 is a perspective view of steering control 108 showing an internal opening 3380 that fits onto steering base 730. The steering control 108 further includes a protrusion 3382 that fits into a recess in the steering base 730, thereby preventing rotation of the steering control 108. In addition, an opening 3384 is formed in the protrusion 3382, and this opening secures the cable connecting to the articulating tip of the stylet; FIG. 34 is an end view of the steering control 108; and FIG. 35 is a side view of the steering control 108.

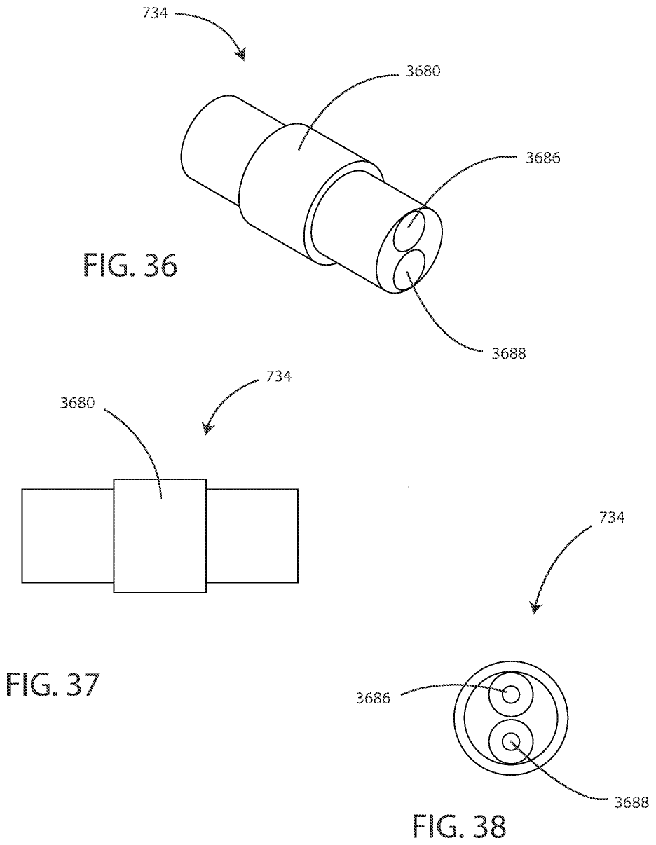

FIG. 36 is a perspective view of an articulation collar 3680 of a stylet in accordance with an embodiment; and FIG. 37 is a side view of the articulation collar 734; FIG. 38 is an end view of the articulation collar 3680, having pathways 3686 and 3688.

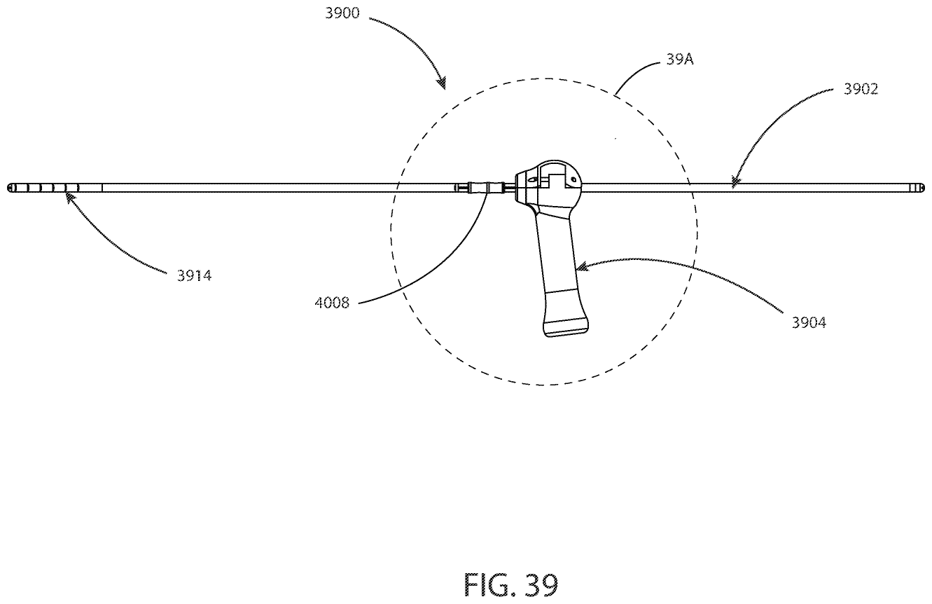

FIG. 39 is a drawing showing a side plan view of endotracheal intubation tool 3900 for assisting in placement of an endotracheal tube made in accordance with an example embodiment. The intubation tool 3900 includes a stylet 3902 and a handle 3904 having an articulating tip 3914 and a steering control 4008. The steering control 4008 slides forward and backward (distally and proximally) along the stylet 3902, and this movement of the steering control 4008 moves the tip of the stylet upward and downward (dorsally and ventrally).

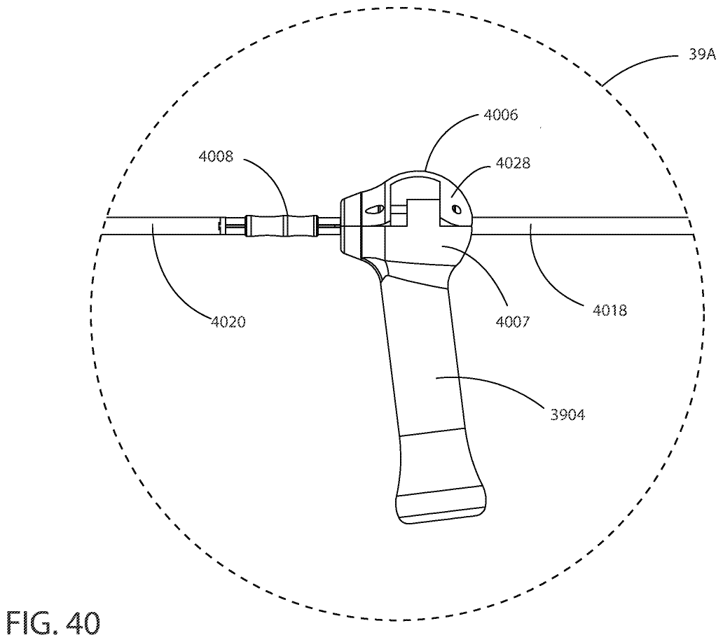

FIG. 40 is closeup of the endotracheal intubation tool of FIG. 39, enlarging the portion from dotted circle 39A. FIG. 40 shows the distal end 4020 of the stylet and the proximal end 4018 of the stylet, as well as clasp 4006 on handle 3904, along with base 4007 of handle 3904. The stylet is secured between the clasp 4006 and the base 4007 during placement of the stylet into a patient. After placement of the stylet into the patient the clasp is opened or removed, freeing the stylet from the handle 3904 so that an intubation tube may be inserted over the stylet. During use rounded surface 4028 of the handle 3904 is typically placed within the palm of the medical professional performing the intubation procedure, and the medical professional fingers wrap around the top of the handle 3904 to grasp the steering control 4008. Typically 2 or 3 fingers make contact with the steering control 4008 so as to pinch the steering control 4008 between the fingers. Minimal force is thereafter needed to move the steering control forward and backward to articulate the tip of the stylet (not shown). Thus, merely drawing the fingers forward and backward as they contact the stylet, typically in a pinching arrangement with two or more fingers arranged around the steering control 4008, results in articulation of the tip of the stylet.

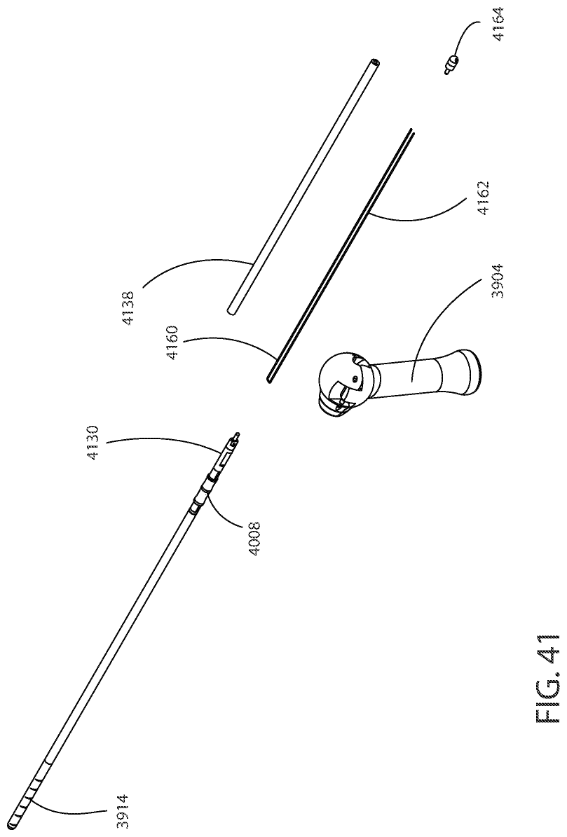

FIG. 41 is a drawing showing an exploded view of components of an endotracheal intubation tool for assisting in placement of an endotracheal tube made in accordance with an example embodiment. The components include handle 3904, articulating tip 3914, steering control 4008 positioned on a steering base 4130. In the depicted embodiment, two lengths of cable 4160 and 4162 which extend through the proximal end of the stylet through proximal sleeve 4138. Tip 4164 is at the most proximal end of the stylet.

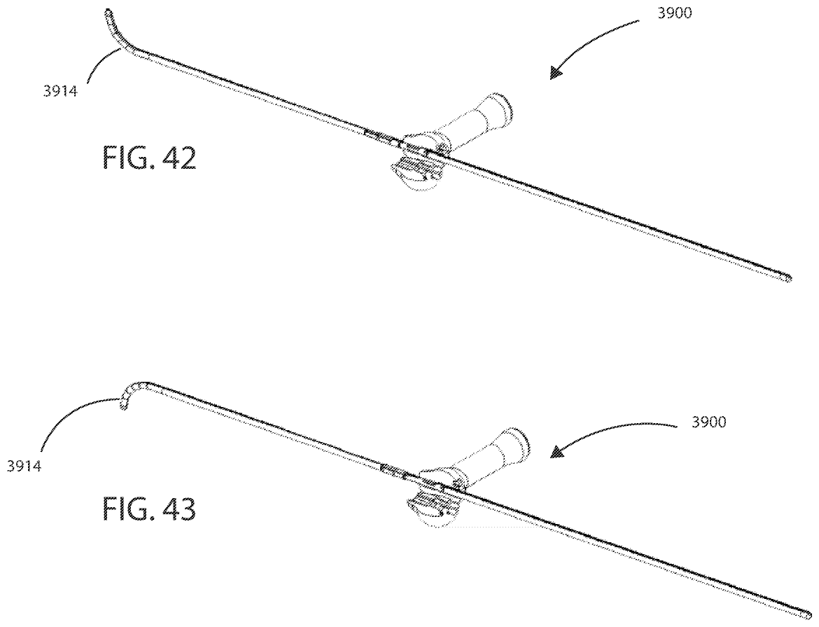

FIG. 42 is a drawing showing a side perspective view of endotracheal intubation tool 3900 for assisting in placement of an endotracheal tube made in accordance with an example embodiment, showing the articulating tip in an upward (or dorsal) articulation. In the embodiment shown the articulating tip 3914 is shown in a position where the tip is articulated in a dorsal (or upward) orientation. In the depicted embodiment the upward orientation occurs as the steering control 4008 (see, e.g., FIGS. 40 and 41) is drawn in a proximal direction. FIG. 43 is a drawing showing a side perspective view of the endotracheal intubation tool of FIG. 42, with the articulating tip 3914 in a ventral (or downward) orientation. In the depicted embodiment the downward orientation occurs as the steering control 4008 is moved in distal direction.

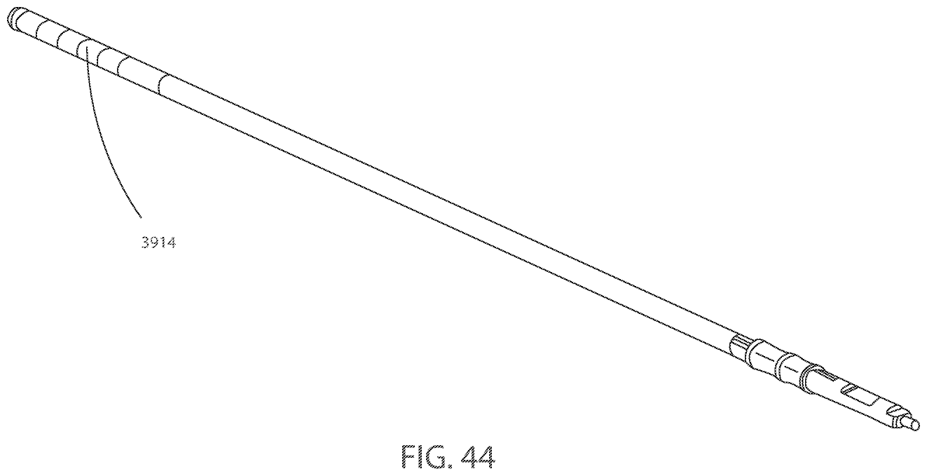

FIG. 44 is drawing showing the articulating tip 3914 of an endotracheal intubation tool in a neutral position. In general, the articulating tip 3914 is in a neutral (or straight) position when no forced is being applied to the tip, such as from the steering control 4008.

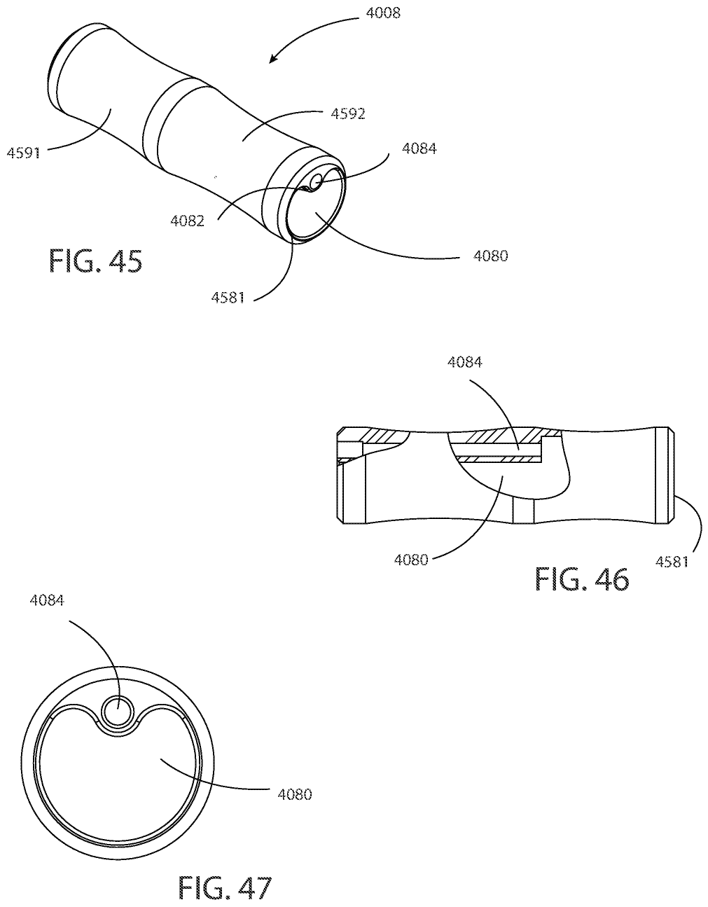

FIG. 45 is a perspective view of steering control 4008 of an intubation tool stylet in accordance with an embodiment, the steering control 4008. Steering control 4008 has internal opening 4080 that fits onto steering base. The steering control 4008 further includes a protrusion 4082 that fits into a recess in the steering base, thereby preventing rotation of the steering control 108. Steering control 4008 also includes an opening 4084 for securing the cable connected to the articulating tip of the stylet. The steering control 4008 has to recessed portions 4591 and 4592 for gripping by fingers (although other configurations are possible). Lip 4581 of the steering control 4008 is also shown. FIG. 46 is a side view of steering control 4008 of an intubation tool stylet in accordance with an embodiment, with a partial cross section. FIG. 47 is an end view of steering control 4008 of an intubation tool stylet in accordance with an embodiment.

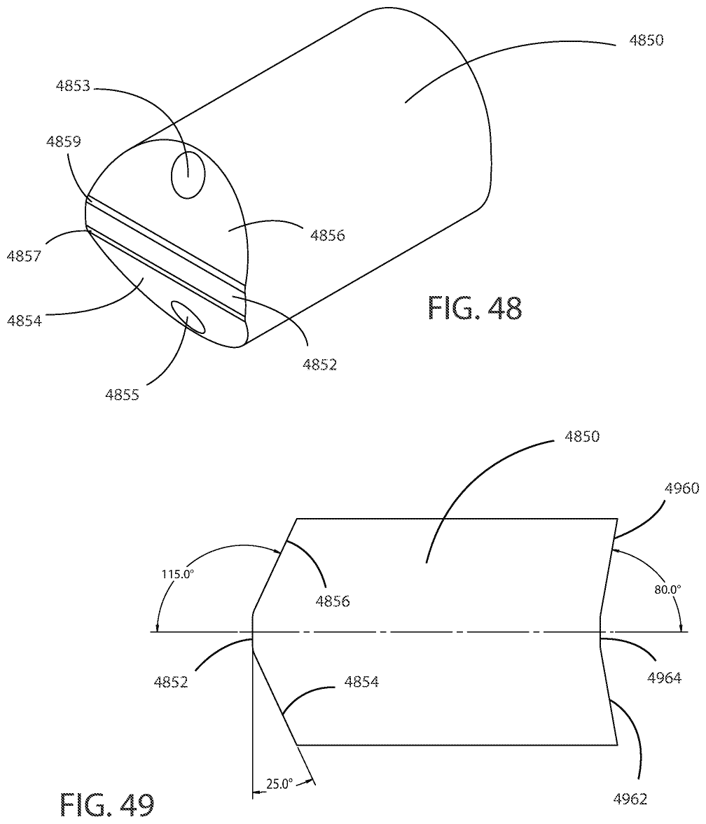

FIG. 48 is a perspective view of a vertebrate 4850 from the articulating end of an articulating tip, showing the vertebrate 4850 independent of other components. The vertebrate 4850 has a first distal angled face 4854 and a second distal angled face 4856 separated, in this embodiment, by a distal flat portion 4852, along with two optional transition regions 4857 and 4859. The vertebrate also includes internal pathways 4853 and 4855 through which the cables are passed to secure multiple vertebrate together and to apply tension to articulate the vertebrate relative to one another.

FIG. 49 is a side view of a vertebrate 4850 from the articulating end of an articulating tip, showing the vertebrate independent of other components. The first angled face 4854 and second angled face 4856 are shown, along with flat portion 4852. These distal angled faces 4854, 4856 and distal flat portion 4852 are on the distal end of the vertebrate (that portion closest to the patient). FIG. 49 further shows the proximal end of the vertebrate, including a first proximal angled face 4960 and a second proximal angled face 4962, and a proximal flat portion 4964.

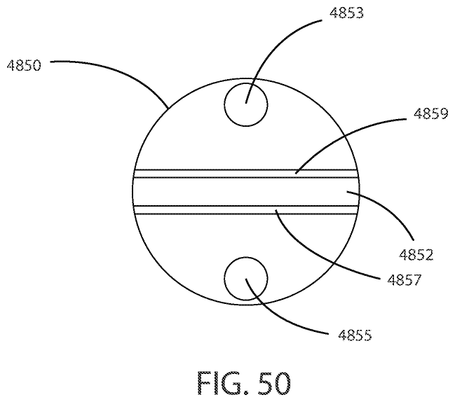

FIG. 50 is a distal end view of a vertebrate 4850 from the articulating end of an articulating tip, showing the vertebrate 4850 independent of other components. The vertebrate 4850 has a first distal angled face 4854 and a second distal angled face 4856 separated, in this embodiment, by a distal flat portion 4852, along with two optional transition regions 4857 and 4859. The vertebrate also includes internal pathways 4853 and 4855 through which the cables are passed to secure multiple vertebrate together and to apply tension to articulate the vertebrate relative to one another.

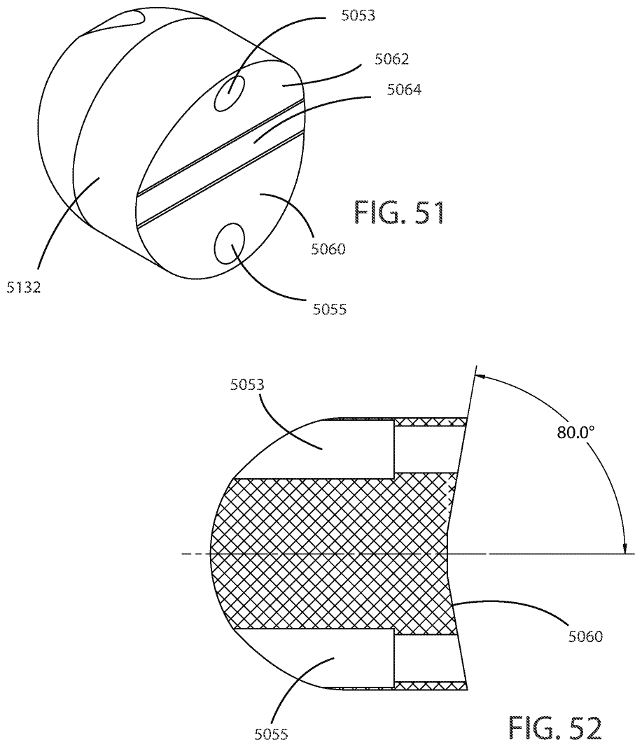

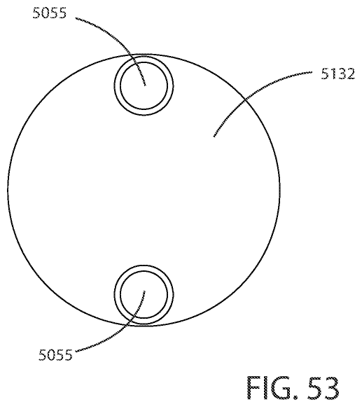

FIG. 51 is a perspective view of the tip member 5132 from an end of an articulating tip, showing the tip member 5060 independent of other components. The tip member 5132 has a first proximal angled face 5060 and second proximal angled face 5062, plus a proximal flat portion 5064. Passageways 5053 and 5055 allow for placement of ends of the control cable (not shown) which are typically secured by adhesive or other means in the end of the passageways. FIG. 52 is a side cross-sectional view of the tip member 5060 from an articulating end of an articulating tip, showing the tip member independent of other components, and FIG. 53 is an end view of the tip member 5060.

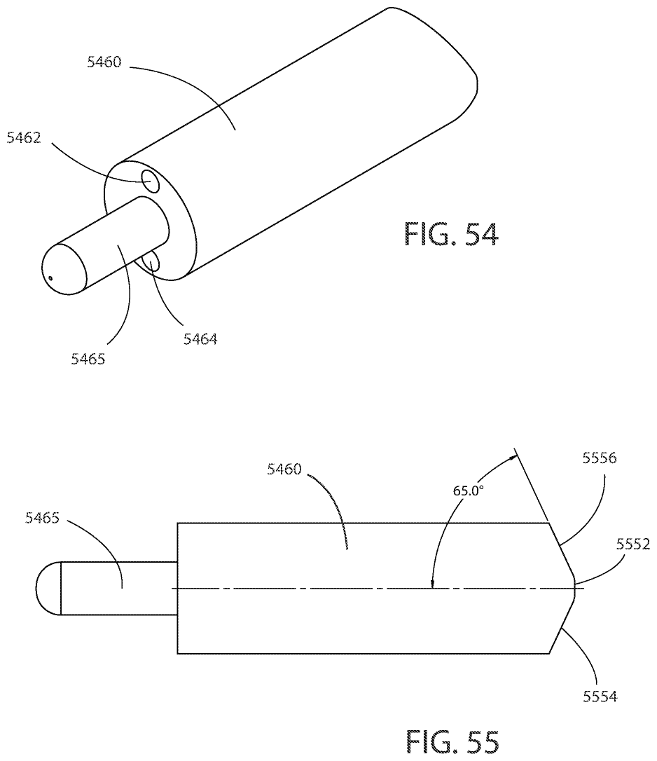

FIG. 54 is a perspective view of a base member 5460 from the proximal end of an articulating tip, showing the base member independent of other components; and FIG. 55 is a side view of the base member 5460. The base member 5460 includes passageways 5462 and 5464, and also include a stem 5465 that can fit within a hole in the shaft of the stylus to secure it into place. The distal end of the base member 5460 includes distal angled face 5554 and second distal angled face 5556, separated by a distal flat portion 5552

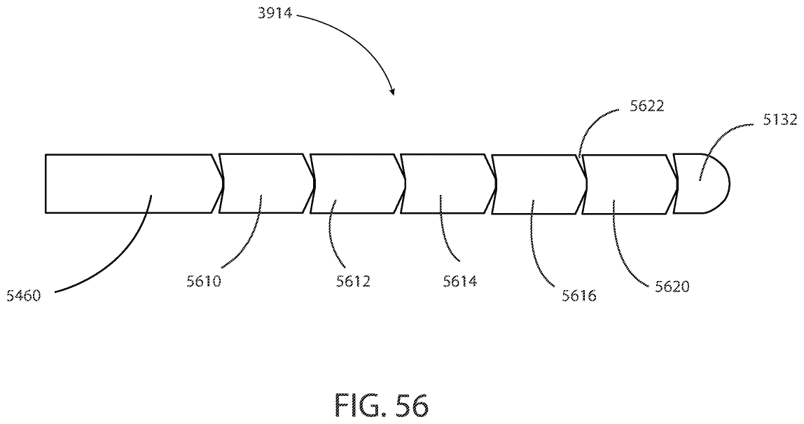

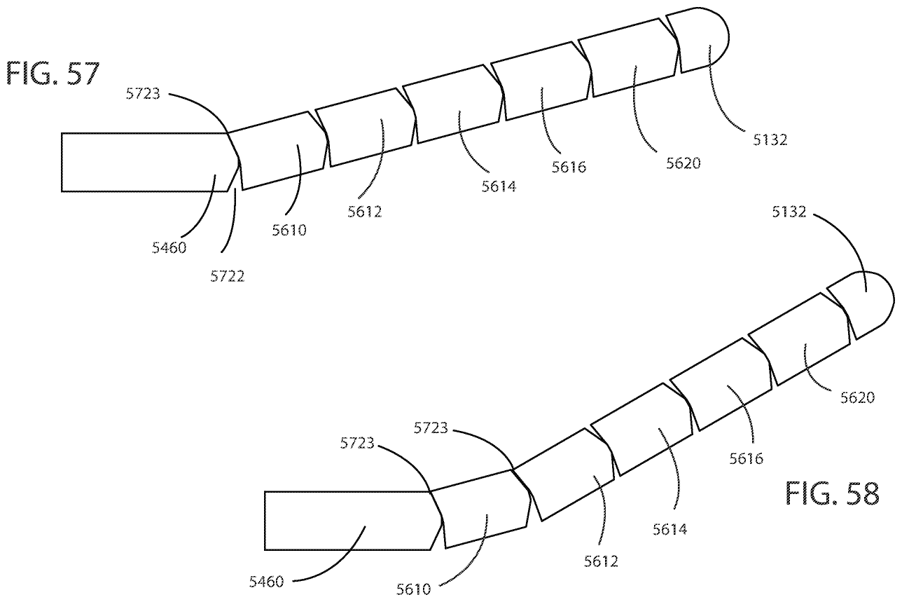

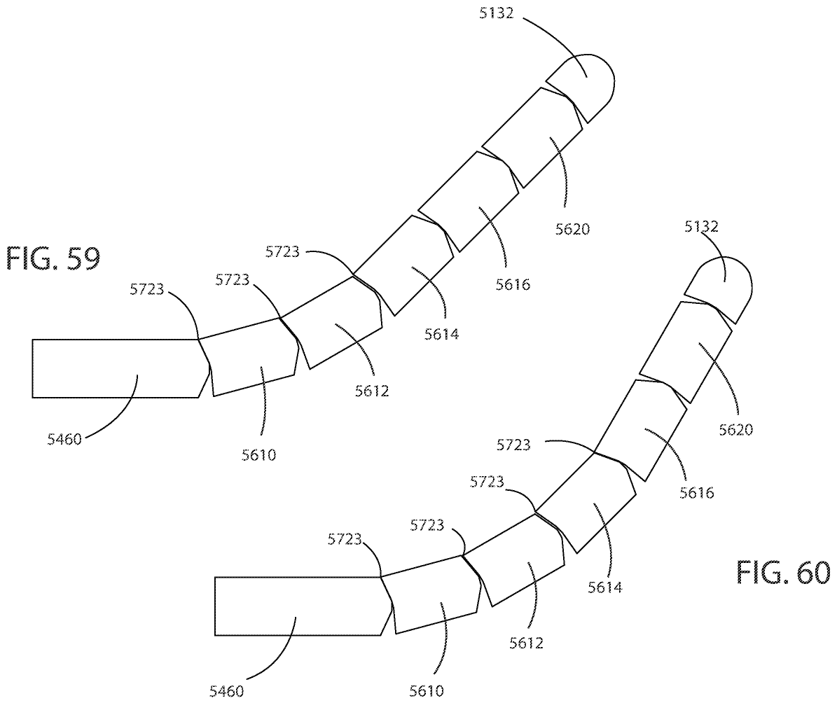

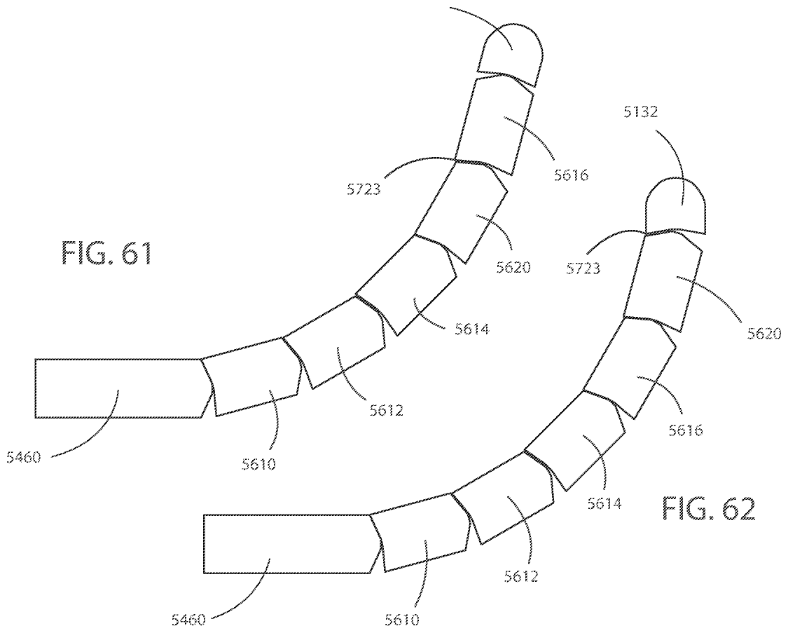

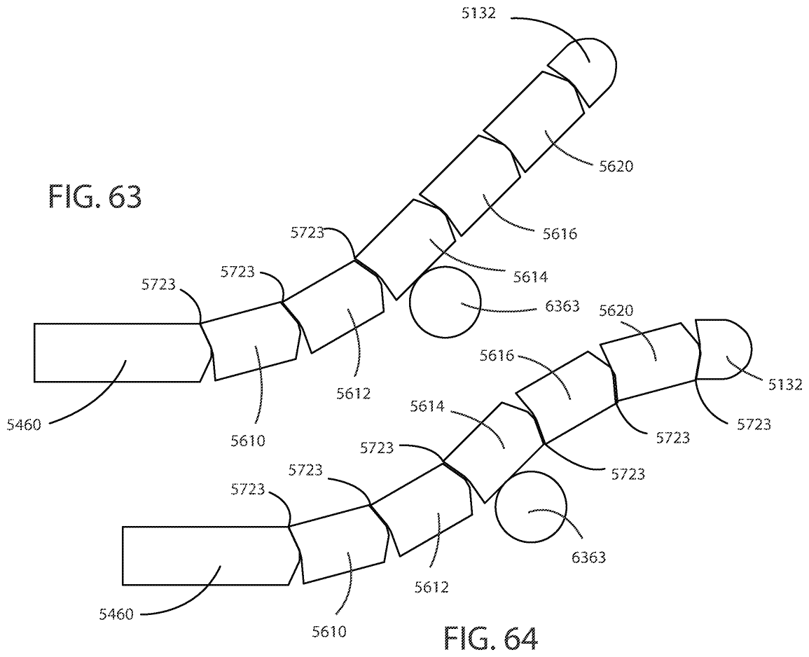

FIG. 56 is a side elevational view of components of an articulating tip of an endotracheal intubation tool in a neutral position, showing a base member 5460, multiple vertebrate 5610, 5612, 5614, 5616, and 5620, along with a tip member 5132. A gap 5622 is shown between the vertebrate 5616 and 5620. FIGS. 57 to 62 show the articulating tip in increasing levels of articulation. As the vertebrate start to articulate the gaps 5622 close into contact regions 5723. On the opposite side an opening 5722 becomes larger. The proximal vertebrate articulate first, followed sequentially down the articulating tip to the most distal vertebrate and the tip member 5132. FIGS. 63 and 64 show the articulating around a stylized obstruction 6363, such as a portion of a patient's anatomy.

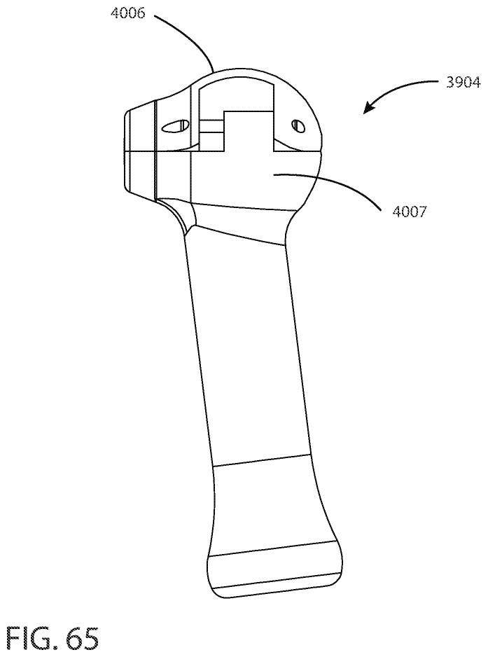

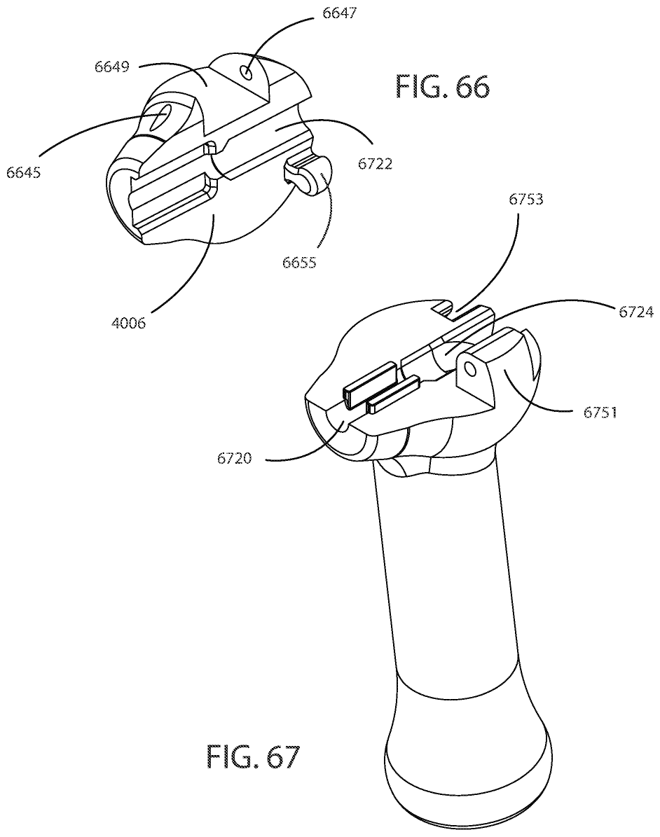

FIG. 65 is a side view of a handle 3904 of an endotracheal intubation tool with the stylet of the tool removed, showing a base 4007 and clasp 4006. FIG. 66 is a perspective view of a clasp 4006 of a handle, showing a protrusion 6655 for fitting into a recess 6753 of a base of a handle shown in FIG. 67 to snap the clasp and base together. A recess 6722 in the clasp combines with a recess 6720 in the base to form a passageway through the handle for the stylet. A hinge for the clasp and base is formed from an elevated portion 6751 of the base that fits into a slot 6649 in the clasp, and a pin can be inserted through holes 6645 and 6647 through the elevated portion 6751 of the base. Also shown is a recess 6724 that allows for the stylet to be bent downward and out of the way of the user while gripping the handle 3904

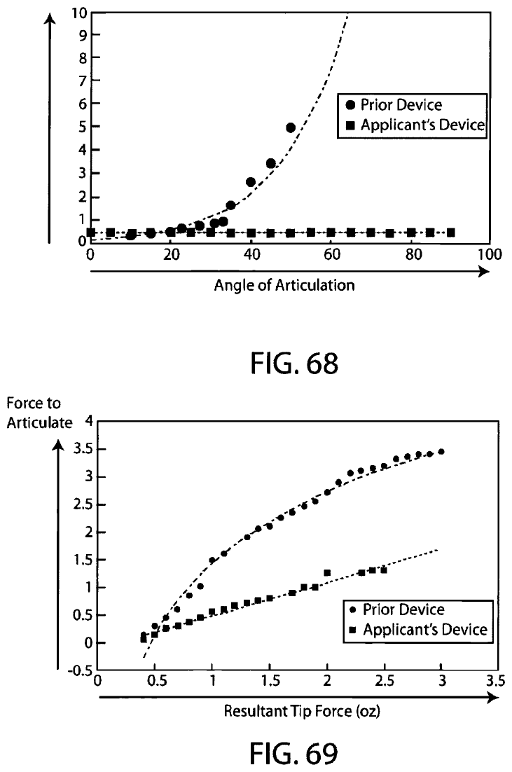

FIG. 68 is a graph showing force relative to articulation position of an articulating tip of an endotracheal intubation tool.

FIG. 69 is a graph showing force relative to articulation position of an articulating tip of an endotracheal intubation tool.

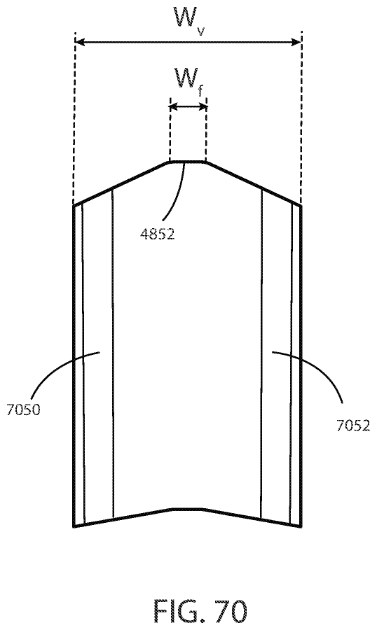

FIG. 70 is a side cross-sectional of a vertebrate from an articulating end of an articulating tip, showing passageways 7050 and 7052. The vertebrate has a total width of Wᵥ, and the distal flat portion 4852 has a width of W_{f}.

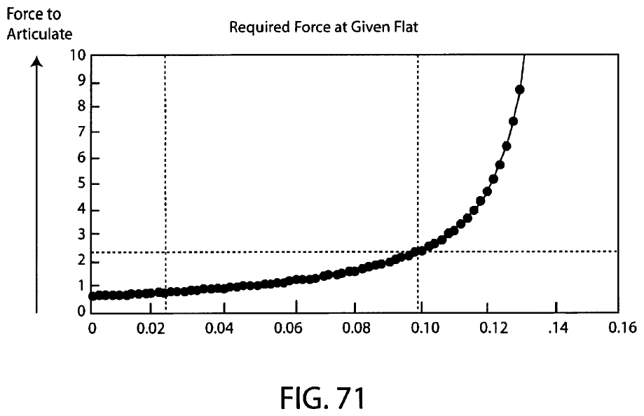

FIG. 71 is a graph showing required to articulate a vertebrate relative to flat portion of the vertebrate.

In an embodiment, a intubation tool for assisting in placement of an endotracheal tube, the intubation tool is included having a) a stylet having a proximal end, a distal end, and a medial location, the stylet: i) configured for receiving an endotracheal tube, and ii) having an articulating distal tip, the articulating distal tip being configured to articulate between more than one shape, and b) a removable handle secured to a medial location on of the stylet.

In an embodiment, wherein articulation of the distal tip of the stylet can be accomplished with a single hand that holds the removable handle and articulates the distal end.

In an embodiment, wherein articulation of the distal tip of the stylet can be accomplished with a single finger manipulating a steering control mounted on the stylet.

In an embodiment, wherein articulation of the distal tip of the stylet can be accomplished with a two fingers manipulating a steering control mounted on the stylet, the steering control sliding along the stylet.

In an embodiment, wherein articulation of the distal end of the stylet can be accomplished with three fingers from a single hand that articulates the distal end.

In an embodiment, the articulating distal tip can be articulated with a force of less than 9 Newton.

In an embodiment, the articulating distal tip can be articulated with a force of 4 to 9 Newton.

In an embodiment, the articulating distal tip is deformable upon contact with an obstruction.

In an embodiment, the articulating distal tip transmits force back to steering control upon contact with an obstruction.

In an embodiment, wherein translation movement of a steering control results in bending articulation of the articulating distal tip.

In an embodiment, wherein translation movement of a steering control by a distance D results in bending articulation of the distal tip by a distance of at least 150 percent of D.

In an embodiment, wherein translation movement of the steering control in a first direction results in bending articulation of the articulating distal tip in a first direction, and translational movement of the steering control in an opposite direction results in bending articulation of the articulating distal tip in a direction opposite the first direction.

In an embodiment, the stylet includes tubing containing at least one lumen.

In an embodiment, the stylet includes bilumen tubing.

In an embodiment, wherein upon removal of the handle from the medial portion of the stylet, the medial portion of the stylet has a diameter no greater than 120 percent of the remainder of the stylet.

In an embodiment, the widest portion of the stylet after removal of the handle is less than the diameter of the interior of the lumen of an endotracheal tube to be installed on the stylet.

In an embodiment, wherein handle and stylet provide a clear sight line of angle α of 20 degrees along a dorsal elevation L_{D} of 1 cm.

In an embodiment, wherein handle and stylet provide a clear sight line of angle α of 20 degrees along a dorsal elevation L_{D} of 2 cm.

In an embodiment, wherein handle and stylet provide a clear sight line of angle α of 20 degrees along a dorsal elevation L_{D} of 3 cm.

In an embodiment, the handle and stylet provide a clear sight line of angle α of 90 degrees along a dorsal elevation L_{D} of 1 cm.

In an embodiment, the handle and stylet provide a clear sight line of angle α of 180 degrees along a dorsal elevation L_{D} of 3 cm.

In an embodiment, the handle and stylet provide a clear sight line of angle α of 270 degrees along a dorsal elevation L_{D} of 3 cm.

In an embodiment, the stylet has a length at least 1.5 times the length of an endotracheal tube to be installed on the stylet.

In an embodiment, the stylet has a length at least 2.0 times the length of an endotracheal tube to be installed on the stylet.

In an embodiment, wherein handle and stylet provide a substantially clear sight-line along the length of the stylet when the stylet is in a non-articulated mode.

In an embodiment, wherein handle and stylet provide a substantially clear sight line of at least 180 degrees along the dorsal line of the handle and stylet combination.

In an embodiment, wherein handle and stylet provide a substantially clear sight line of at least 90 degrees along a dorsal line of the handle and stylet combination, within 1 cm of the central axis of the stylet.

In an embodiment, wherein handle and stylet provide a substantially clear sight line of at least 90 degrees along a dorsal line of the handle and stylet combination, within 2 cm of the central axis of the stylet.

In an embodiment, wherein handle and stylet provide a substantially clear sight line of at least 270 degrees along a dorsal line of the handle and stylet combination, within 1 cm of the central axis of the stylet.

In an embodiment, the handle is removable from the medial location of the stylet to allow placement of an endotracheal tube in place.

In an embodiment, the stylet articulates in a plane.

In an embodiment, the plane in which the stylet articulates can be selected by rotation the handle.

In an embodiment, the stylet includes an internal mechanism for articulating the distal end.

In an embodiment, the stylet includes an internal cable connected to the distal end, and wherein pulling on the cable from the medial location results in articulating the distal end of the stylet.

In an embodiment, further can include a steering element.

In an embodiment, the steering element includes a tube surrounding a portion of the medial location of the stylet.

In an embodiment, a method for placement of an endotracheal tube, the method is included, the method a) providing an intubation tool for assisting in placement of an endotracheal tube, the intubation tool is included, the method i) a stylet having a proximal end, distal end, and intermediate medial portion, the stylet configured for receiving an endotracheal tube, and having an articulating distal tip, the articulating distal tip being configured to articulate between more than one shape using one to three fingers, and ii) a handle secured to a medial position on the stylet, b) inserting the distal end of the stylet of the intubation tool into the trachea of a person, including articulating the distal end of the stylet during insertion to aid in passage through the larynx, c) removing the handle from the medial portion of the stylet, d) placing an endotracheal tube over the medial location of the stylet and down toward the distal end of the stylet and into the trachea of the person, and e) removing the stylet from the patient's trachea while keeping the endotracheal tube in place.

In an embodiment, the stylet articulates in a plane.

In an embodiment, the stylet includes an internal mechanism for articulating the distal tip.

In an embodiment, the stylet further includes a flexible intermediate portion.

In an embodiment, the stylet includes an internal cable connected to the distal tip, and wherein pulling on the cable from the medial location results in articulating the distal tip of the stylet.

In an embodiment, further can include an actuator.

In an embodiment, the actuator includes a tube surrounding a portion of the medial location of the stylet.

In an embodiment, wherein handle and stylet provide a substantially clear sight-line along the length of the stylet when the stylet is in a non-articulated mode.

In an embodiment, wherein handle and stylet provide a substantially clear sight line of at least 180 degrees along the dorsal line of the handle and stylet combination.

In an embodiment, wherein handle and stylet provide a substantially clear sight line of at least 90 degrees along a dorsal line of the handle and stylet combination, within 1 cm of the central axis of the stylet.

In an embodiment, wherein handle and stylet provide a substantially clear sight line of at least 90 degrees along a dorsal line of the handle and stylet combination, within 2 cm of the central axis of the stylet.

In an embodiment, wherein handle and stylet provide a substantially clear sight line of at least 270 degrees along a dorsal line of the handle and stylet combination, within 1 cm of the central axis of the stylet.

In an embodiment, a intubation tool for assisting in placement of an endotracheal tube, the endotracheal intubation tool is included having a) a stylet having a medial location and a distal end, the stylet: i.) configured for receiving an endotracheal tube, and ii) having an articulating distal tip, the articulating tip end being configured to articulate between more than one shape, and b) a handle secured to the medial location of the stylet, wherein actuation is provided by a cable.

In an embodiment, the cable radial location optimized to facilitate articulation and minimize pull force.

In an embodiment, the handle is removable from the stylet.

In an embodiment, the handle is removable from the medial location of the stylet to allow placement of an endotracheal tube in place.

In an embodiment, the stylet articulates in a plane.

In an embodiment, the stylet includes an internal mechanism for articulating the distal end.

In an embodiment, where the stylet further includes a flexible intermediate portion.

In an embodiment, the stylet includes an internal cable connected to the distal end, and wherein pulling on the cable from the medial location results in articulating the distal end of the stylet.

In an embodiment, further can include an actuator.

In an embodiment, the actuator includes a tube surrounding a portion of the medial location of the stylet.

In an embodiment, a method for placement of an endotracheal tube, the method is included, the method a) providing an intubation tool for assisting in placement of an endotracheal tube, the intubation tool is included, the method i) a stylet having a medial location and a distal end, the stylet configured for receiving an endotracheal tube, and having an articulating distal end, the articulating distal end being configured to articulate between more than one shape, and ii) a handle secured to the medial location of the stylet, b) inserting the distal end of the stylet of the endotracheal intubation tool into the trachea of a person, including articulating the distal end of the stylet during insertion to aid in passage through the larynx, c) removing the handle from the medial location of the stylet, d) inserting an endotracheal tube over the medial location of the stylet and down toward the distal end of the stylet, and e) removing the stylet from the patient's trachea while keeping the endotracheal tube in place, wherein the actuation can be performed with a single hand.

In an embodiment, the endotracheal intubation tool for assisting in placement of an endotracheal tube is removable from the medial location of the stylet.

In an embodiment, wherein actuation occurs push/pull action on the tip by allowing three finger, 360 degree contact of circular trigger.

In an embodiment, the endotracheal intubation stylet includes an internal mechanism for articulating the distal end.

In an embodiment, the stylet further includes a flexible intermediate portion.

In an embodiment, the stylet includes an internal cable connected to the distal end, and wherein pulling on the cable from the medial location results in articulating the distal end of the stylet.

In an embodiment, further can include an actuator.

In an embodiment, the actuator includes a tube surrounding a portion of the medial location of the stylet.

In an embodiment, a intubation tool for assisting in placement of an endotracheal tube, the intubation tool is included having a) a stylet having a proximal end, a distal end, and a medial location, the stylet: i) configured for receiving an endotracheal tube, and ii) having an articulating distal tip, the articulating distal tip being configured to articulate between more than one shape, and b) a removable handle secured to a medial location on of the stylet, wherein the stylet has a substantially uniform outer diameter.

In an embodiment, the minimum non-tip diameter of stylet is within 70 percent of the maximum non-tip diameter of the stylet.

In an embodiment, the minimum non-tip diameter of stylet is within 80 percent of the maximum non-tip diameter of the stylet.

In an embodiment, the minimum non-tip diameter of stylet is within 90 percent of the maximum non-tip diameter of the stylet.

In an embodiment, the stylet includes a keyed surface for securing a handle.

In an embodiment, a intubation tool for assisting in placement of an endotracheal tube, the intubation tool is included having a) a stylet having a proximal end, a distal end, and a medial location, the stylet: i) configured for receiving an endotracheal tube, and ii) having an articulating distal tip, the articulating distal tip being configured to articulate between more than one shape, and b) a removable handle secured to a medial location on of the stylet wherein the stylet includes a flexible cable to articulate the distal tip.

In an embodiment, the distal end of the stylet is in a neutral position when no articulating force is applied.

In an embodiment, the force to articulate the distal end is less than 2 pounds.

In an embodiment, the force to articulate the distal end is less than 4 pounds.

In an embodiment, the force to articulate the distal end from 60 to 90 degrees is within 150 percent of the force to articulate the distal end from 0 to 30 degrees.

In an embodiment, the force to articulate the distal end from 80 to 90 degrees is within 150 percent of the force to articulate the distal end from 0 to 10 degrees.

In an embodiment, a intubation tool for assisting in placement of an endotracheal tube, the intubation tool is included having a) a stylet having a proximal end, a distal end, and a medial location, the stylet: i) configured for receiving an endotracheal tube, and ii) having an articulating distal tip, the articulating distal tip being configured to articulate between more than one shape, and the articulating distal tip includes a plurality of free vertebrate; and b) a removable handle secured to a medial location on of the stylet.

In an embodiment, the vertebrate have a substantially flat peak

In an embodiment, the vertebrate have a substantially flat valley

In an embodiment, the vertebrate interface angle is from 10 to 20 degrees.

In an embodiment, wherein handle and stylet provide a substantially clear sight line of at least 90 degrees along a dorsal line of the handle and stylet combination, within 1 cm of the central axis of the stylet.

In an embodiment, wherein handle and stylet provide a substantially clear sight-line along the length of the stylet when the stylet is in a non-articulated mode.

In an embodiment, the articulating distal tip can be articulated with a force of 4 to 9 Newton.

In an embodiment, the handle of the tool includes a grip extension oriented at 90 degrees to the plane of movement of the distal end of the stylus.

In an embodiment, the distal end of the stylet is in a neutral position when no articulating force is applied.

In an embodiment, a intubation tool for assisting in placement of an endotracheal tube, the intubation tool is included having a) a stylet having a proximal end, a distal end, and a medial location, the stylet: i) configured for receiving an endotracheal tube, and ii) having an articulating distal tip, the articulating distal tip being configured to articulate between more than one shape, and b) a removable handle secured to a medial location on of the stylet, wherein the articulating tip is not rigid.

In an embodiment, the force to articulate the distal end from 60 to 90 degrees is within 150 percent of the force to articulate the distal end from 0 to 30 degrees.

In an embodiment, wherein handle and stylet provide a substantially clear sight line of at least 90 degrees along a dorsal line of the handle and stylet combination, within 1 cm of the central axis of the stylet.

In an embodiment, the articulating distal tip can be articulated with a force of 4 to 9 Newton.

In an embodiment, wherein handle and stylet provide a substantially clear sight-line along the length of the stylet when the stylet is in a non-articulated mode.

In an embodiment, the handle of the tool includes a grip extension oriented at 90 degrees to the plane of movement of the distal end of the stylus.

In an embodiment, a intubation tool for assisting in placement of an endotracheal tube, the intubation tool is included having a) a stylet having a proximal end, a distal end, and a medial location, the stylet: i) configured for receiving an endotracheal tube, and ii) having an articulating distal tip, the articulating distal tip being configured to articulate between more than one shape, and b) a removable handle secured to a medial location on of the stylet, wherein the articulating tip transmits tactile feedback. In an embodiment, the force to articulate the distal end is less than 2 pounds.

In an embodiment, the stylet includes an internal cable connected to the distal end, and wherein pulling on the cable from the medial location results in articulating the distal end of the stylet.

In an embodiment, wherein handle and stylet provide a substantially clear sight-line along the length of the stylet when the stylet is in a non-articulated mode.

In an embodiment, wherein handle and stylet provide a substantially clear sight line of at least 90 degrees along a dorsal line of the handle and stylet combination, within 1 cm of the central axis of the stylet.

In an embodiment, the force to articulate the distal end from 60 to 90 degrees is within 150 percent of the force to articulate the distal end from 0 to 30 degrees.

In an embodiment, the distal end of the stylet is in a neutral position when no articulating force is applied.

In an embodiment, a intubation tool for assisting in placement of an endotracheal tube, the intubation tool is included having a stylet having a proximal end, a distal end, and a medial location, the stylet: i) configured for receiving an endotracheal tube, ii) having an articulating distal tip, the articulating distal tip being configured to articulate between more than one shape, and iii) a slidable actuator articulating the distal tip, the slidable actuator being engageable around at least 180 degree of the central axis of the stylet.

In an embodiment, the slidable actuator is engageable around at least 270 of the central axis of the stylet.

In an embodiment, the slidable actuator is engageable around at 360 of the central axis of the stylet.

In an embodiment, a intubation tool for assisting in placement of an endotracheal tube, the intubation tool is included having a) a stylet having a proximal end, a distal end, and a medial location, the stylet including a bi-lumen extrusion, the stylet: i) configured for receiving an endotracheal tube, and ii) having an articulating distal tip, the articulating distal tip being configured to articulate between more than one shape, and b) a removable handle secured to a medial location on of the stylet.

In an embodiment, the force to articulate the distal end from 60 to 90 degrees is within 150 percent of the force to articulate the distal end from 0 to 30 degrees.

In an embodiment, wherein handle and stylet provide a substantially clear sight-line along the length of the stylet when the stylet is in a non-articulated mode.

In an embodiment, the articulating distal tip can be articulated with a force of 4 to 9 Newton.

In an embodiment, the handle of the tool includes a grip extension oriented at 90 degrees to the plane of movement of the distal end of the stylus.

In an embodiment, wherein handle and stylet provide a substantially clear sight line of at least 90 degrees along a dorsal line of the handle and stylet combination, within 1 cm of the central axis of the stylet.

In an embodiment, the distal end of the stylet is in a neutral position when no articulating force is applied.

In an embodiment, a method for placement of an endotracheal tube, the method is included, the method a) providing an intubation tool for assisting in placement of an endotracheal tube, the intubation tool is included, the method i) a stylet having a proximal end, distal end, and intermediate medial portion, the stylet configured for receiving an endotracheal tube, and having an articulating distal tip, the articulating distal tip being configured to articulate between more than one shape using one to three fingers, and ii) a handle secured to a medial position on the stylet, b) inserting the distal end of the stylet of the intubation tool into the trachea of a person, including articulating the distal end of the stylet during insertion to aid in passage through the larynx, c) removing the handle from the medial portion of the stylet, d) placing an endotracheal tube over the medial location of the stylet and down toward the distal end of the stylet and into the trachea of the person, and e) removing the stylet from the patient's trachea while keeping the endotracheal tube in place.

In an embodiment, the force to articulate the distal end from 60 to 90 degrees is within 150 percent of the force to articulate the distal end from 0 to 30 degrees.

In an embodiment, the stylet includes an internal cable connected to the distal end, and wherein pulling on the cable from the medial location results in articulating the distal end of the stylet.

In an embodiment, the articulating distal tip can be articulated with a force of 4 to 9 Newton.

In an embodiment, the handle of the tool includes a grip extension oriented at 90 degrees to the plane of movement of the distal end of the stylus.

In an embodiment, a intubation tool for assisting in placement of an endotracheal tube, the endotracheal intubation tool is included having a) a stylet having a medial location and a distal end, the stylet: i.) configured for receiving an endotracheal tube, and ii) having an articulating distal tip, the articulating tip end being configured to articulate between more than one shape, and b) a handle secured to the medial location of the stylet, wherein actuation is provided by a cable.

It should be noted that, as used in this specification and the appended claims, the singular forms “a,” “an,” and “the” include plural referents unless the content clearly dictates otherwise. It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

We claim:

1. An intubation tool for assisting in placement of an endotracheal tube, the intubation tool comprising:
a) a stylet having a proximal end, a distal end, and a medial location, the stylet:
i) configured for receiving an endotracheal tube, and
ii) having an articulating distal tip, the articulating distal tip being configured to articulate between more than one shape; and
b) a removable handle secured to a medial location on of the stylet.

2. The intubation tool for assisting in placement of an endotracheal tube of any of claims 1 and 3-36, wherein articulation of the distal tip of the stylet can be accomplished with a single hand that holds the removable handle and articulates the distal end.

3. The intubation tool for assisting in placement of an endotracheal tube of any of claims 1-2 and 4-36, wherein articulation of the distal tip of the stylet can be accomplished with a single finger manipulating a steering control mounted on the stylet.

4. The intubation tool for assisting in placement of an endotracheal tube of any of claims 1-3 and 5-36, wherein articulation of the distal tip of the stylet can be accomplished with a two fingers manipulating a steering control mounted on the stylet, the steering control sliding along the stylet.

5. The intubation tool for assisting in placement of an endotracheal tube of any of claims 1-4 and 6-36, wherein articulation of the distal end of the stylet can be accomplished with three fingers from a single hand that articulates the distal end.

6. The intubation tool for assisting in placement of an endotracheal tube of any of claims 1-5 and 7-36, wherein the articulating distal tip can be articulated with a force of less than 9 Newton.

7. The intubation tool for assisting in placement of an endotracheal tube of any of claims 1-6 and 8-36, wherein the articulating distal tip can be articulated with a force of 4 to 9 Newton.

8. The intubation tool for assisting in placement of an endotracheal tube of any of claims 1-7 and 9-36, wherein the articulating distal tip is deformable upon contact with an obstruction.

9. The intubation tool for assisting in placement of an endotracheal tube of any of claims 1-8 and 10-36, wherein the articulating distal tip transmits force back to steering control upon contact with an obstruction.

10. The intubation tool for assisting in placement of an endotracheal tube of any of claims 1-9 and 11-36, wherein translation movement of a steering control results in bending articulation of the articulating distal tip.

11. The intubation tool for assisting in placement of an endotracheal tube of any of claims 1-10 and 12-36, wherein translation movement of a steering control by a distance D results in bending articulation of the distal tip by a distance of at least 150 percent of D.

12. The intubation tool for assisting in placement of an endotracheal tube of any of claims 1-11 and 13-36, wherein translation movement of the steering control in a first direction results in bending articulation of the articulating distal tip in a first direction; and
translational movement of the steering control in an opposite direction results in bending articulation of the articulating distal tip in a direction opposite the first direction.

13. The intubation tool for assisting in placement of an endotracheal tube of any of claims 1-12 and 14-36, wherein the stylet comprises tubing containing at least one lumen.

14. The intubation tool for assisting in placement of an endotracheal tube of any of claims 1-13 and 15-36, wherein the stylet comprises bilumen tubing.

15. The intubation tool for assisting in placement of an endotracheal tube of any of claims 1-14 and 16-36, wherein upon removal of the handle from the medial portion of the stylet, the medial portion of the stylet has a diameter no greater than 120 percent of the remainder of the stylet.

16. The intubation tool for assisting in placement of an endotracheal tube of any of claims 1-15 and 17-36, wherein the widest portion of the stylet after removal of the handle is less than the diameter of the interior of the lumen of an endotracheal tube to be installed on the stylet.

17. The intubation tool for assisting in placement of an endotracheal tube of any of claims 1-16 and 18-36, wherein handle and stylet provide a clear sight line of angle α of 20 degrees along a dorsal elevation LD of 1 cm.

18. The intubation tool for assisting in placement of an endotracheal tube of any of claims 1-17 and 19-36, wherein handle and stylet provide a clear sight line of angle α of 20 degrees along a dorsal elevation LD of 2 cm.

19. The intubation tool for assisting in placement of an endotracheal tube of any of claims 1-18 and 20-36, wherein handle and stylet provide a clear sight line of angle α of 20 degrees along a dorsal elevation LD of 3 cm.

20. The intubation tool for assisting in placement of an endotracheal tube of any of claims 1-19 and 21-36, wherein the handle and stylet provide a clear sight line of angle α of 90 degrees along a dorsal elevation LD of 1 cm.

21. The intubation tool for assisting in placement of an endotracheal tube of any of claims 1-20 and 22-36, wherein the handle and stylet provide a clear sight line of angle α of 180 degrees along a dorsal elevation LD of 3 cm.

22. The intubation tool for assisting in placement of an endotracheal tube of any of claims 1-21 and 23-36, wherein the handle and stylet provide a clear sight line of angle α of 270 degrees along a dorsal elevation LD of 3 cm.

23. The intubation tool for assisting in placement of an endotracheal tube of any of claims 1-22 and 24-36, wherein the stylet has a length at least 1.5 times the length of an endotracheal tube to be installed on the stylet.

24. The intubation tool for assisting in placement of an endotracheal tube of any of claims 1-23 and 25-36, wherein the stylet has a length at least 2.0 times the length of an endotracheal tube to be installed on the stylet.

25. The intubation tool for assisting in placement of an endotracheal tube of any of claims 1-24 and 26-36, wherein handle and stylet provide a substantially clear sight-line along the length of the stylet when the stylet is in a non-articulated mode.

26. The intubation tool for assisting in placement of an endotracheal tube of any of claims 1-25 and 27-36, wherein handle and stylet provide a substantially clear sight line of at least 180 degrees along the dorsal line of the handle and stylet combination.

27. The intubation tool for assisting in placement of an endotracheal tube of any of claims 1-26 and 28-36, wherein handle and stylet provide a substantially clear sight line of at least 90 degrees along a dorsal line of the handle and stylet combination, within 1 cm of the central axis of the stylet.

28. The intubation tool for assisting in placement of an endotracheal tube of any of claims 1-27 and 29-36, wherein handle and stylet provide a substantially clear sight line of at least 90 degrees along a dorsal line of the handle and stylet combination, within 2 cm of the central axis of the stylet.

29. The intubation tool for assisting in placement of an endotracheal tube of any of claims 1-28 and 30-36, wherein handle and stylet provide a substantially clear sight line of at least 270 degrees along a dorsal line of the handle and stylet combination, within 1 cm of the central axis of the stylet.

30. The intubation tool for assisting in placement of an endotracheal tube of any of claims 1-29 and 31-36, wherein the handle is removable from the medial location of the stylet to allow placement of an endotracheal tube in place.

31. The intubation tool for assisting in placement of an endotracheal tube of any of claims 1-30 and 32-36, wherein the stylet articulates in a plane.

32. The intubation tool for assisting in placement of an endotracheal tube of any of claims 1-31 and 33-36, wherein the plane in which the stylet articulates can be selected by rotation the handle.

33. The intubation tool for assisting in placement of an endotracheal tube of any of claims 1-32 and 34-36, wherein the stylet includes an internal mechanism for articulating the distal end.

34. The intubation tool for assisting in placement of an endotracheal tube of any of claims 1-33 and 35-36, wherein the stylet comprises an internal cable connected to the distal end, and wherein pulling on the cable from the medial location results in articulating the distal end of the stylet.

35. The intubation tool for assisting in placement of an endotracheal tube of any of claims 1-34 and 36, further comprising a steering element.

36. The intubation tool for assisting in placement of an endotracheal tube of any of claims 1-35, wherein the steering element comprises a tube surrounding a portion of the medial location of the stylet.

37. A method for placement of an endotracheal tube, the method comprising:
a) providing an intubation tool for assisting in placement of an endotracheal tube, the intubation tool comprising:
i) a stylet having a proximal end, distal end, and intermediate medial portion, the stylet configured for receiving an endotracheal tube, and having an articulating distal tip, the articulating distal tip being configured to articulate between more than one shape using one to three fingers; and
ii) a handle secured to a medial position on the stylet;
b) inserting the distal end of the stylet of the intubation tool into the trachea of a person, including articulating the distal end of the stylet during insertion to aid in passage through the larynx;
c) removing the handle from the medial portion of the stylet;
d) placing an endotracheal tube over the medial location of the stylet and down toward the distal end of the stylet and into the trachea of the person; and
e) removing the stylet from the patient's trachea while keeping the endotracheal tube in place.

38. The method for placement of an endotracheal tube of any of claims 37 and 39-48, wherein the stylet articulates in a plane.

39. The method for placement of an endotracheal tube of any of claims 37-38 and 40-48, wherein the stylet includes an internal mechanism for articulating the distal tip.

40. The method for placement of an endotracheal tube of any of claims 37-39 and 41-48, wherein the stylet further comprises a flexible intermediate portion.

41. The method for placement of an endotracheal tube of any of claims 37-40 and 42-48, wherein the stylet comprises an internal cable connected to the distal tip, and wherein pulling on the cable from the medial location results in articulating the distal tip of the stylet.

42. The method for placement of an endotracheal tube of any of claims 37-41 and 43-48, further comprising an actuator.

43. The method for placement of an endotracheal tube of any of claims 37-42 and 44-48, wherein the actuator comprises a tube surrounding a portion of the medial location of the stylet.

44. The method for placement of an endotracheal tube of any of claims 37-43 and 45-48, wherein handle and stylet provide a substantially clear sight-line along the length of the stylet when the stylet is in a non-articulated mode.

45. The method for placement of an endotracheal tube of any of claims 37-44 and 46-48, wherein handle and stylet provide a substantially clear sight line of at least 180 degrees along the dorsal line of the handle and stylet combination.

46. The method for placement of an endotracheal tube of any of claims 37-45 and 47-48, wherein handle and stylet provide a substantially clear sight line of at least 90 degrees along a dorsal line of the handle and stylet combination, within 1 cm of the central axis of the stylet.

47. The method for placement of an endotracheal tube of any of claims 37-46 and 48, wherein handle and stylet provide a substantially clear sight line of at least 90 degrees along a dorsal line of the handle and stylet combination, within 2 cm of the central axis of the stylet.

48. The method for placement of an endotracheal tube of any of claims 37-47, wherein handle and stylet provide a substantially clear sight line of at least 270 degrees along a dorsal line of the handle and stylet combination, within 1 cm of the central axis of the stylet.

49. An intubation tool for assisting in placement of an endotracheal tube, the endotracheal intubation tool comprising:
a) a stylet having a medial location and a distal end, the stylet:
i.) configured for receiving an endotracheal tube, and
ii) having an articulating distal tip, the articulating tip end being configured to articulate between more than one shape; and
b) a handle secured to the medial location of the stylet;
wherein actuation is provided by a cable.

50. The intubation tool for assisting in placement of an endotracheal tube of any of claims 49 and 51-58, wherein the cable radial location optimized to facilitate articulation and minimize pull force.

51. The intubation tool for assisting in placement of an endotracheal tube of any of claims 49-50 and 52-58, wherein the handle is removable from the stylet.

52. The intubation tool for assisting in placement of an endotracheal tube of any of claims 49-51 and 53-58, wherein the handle is removable from the medial location of the stylet to allow placement of an endotracheal tube in place.

53. The intubation tool for assisting in placement of an endotracheal tube of any of claims 49-52 and 54-58, wherein the stylet articulates in a plane.

54. The intubation tool for assisting in placement of an endotracheal tube of any of claims 49-53 and 55-58, wherein the stylet includes an internal mechanism for articulating the distal end.

55. The intubation tool for assisting in placement of an endotracheal tube of any of claims 49-54 and 56-58, where the stylet further comprises a flexible intermediate portion.

56. The intubation tool for assisting in placement of an endotracheal tube of any of claims 49-55 and 57-58, wherein the stylet comprises an internal cable connected to the distal end, and wherein pulling on the cable from the medial location results in articulating the distal end of the stylet.

57. The intubation tool for assisting in placement of an endotracheal tube of any of claims 49-56 and 58, further comprising an actuator.

58. The intubation tool for assisting in placement of an endotracheal tube of any of claims 49-57, wherein the actuator comprises a tube surrounding a portion of the medial location of the stylet.

59. A method for placement of an endotracheal tube, the method comprising:
a) providing an intubation tool for assisting in placement of an endotracheal tube, the intubation tool comprising:
i) a stylet having a medial location and a distal end, the stylet configured for receiving an endotracheal tube, and having an articulating distal end, the articulating distal end being configured to articulate between more than one shape; and
ii) a handle secured to the medial location of the stylet;
b) inserting the distal end of the stylet of the endotracheal intubation tool into the trachea of a person, including articulating the distal end of the stylet during insertion to aid in passage through the larynx;
c) removing the handle from the medial location of the stylet;
d) inserting an endotracheal tube over the medial location of the stylet and down toward the distal end of the stylet; and
e) removing the stylet from the patient's trachea while keeping the endotracheal tube in place;
wherein the actuation can be performed with a single hand.

60. The method for placement of an endotracheal tube of any of claims 59 and 61-66, wherein the endotracheal intubation tool for assisting in placement of an endotracheal tube is removable from the medial location of the stylet.

61. The method for placement of an endotracheal tube of any of claims 59-60 and 62-66, wherein actuation occurs push/pull action on the tip by allowing three finger, 360 degree contact of circular trigger.

62. The method for placement of an endotracheal tube of any of claims 59-61 and 63-66, wherein the endotracheal intubation stylet includes an internal mechanism for articulating the distal end.

63. The method for placement of an endotracheal tube of any of claims 59-62 and 64-66, wherein the stylet further comprises a flexible intermediate portion.

64. The method for placement of an endotracheal tube of any of claims 59-63 and 65-66, wherein the stylet comprises an internal cable connected to the distal end, and wherein pulling on the cable from the medial location results in articulating the distal end of the stylet.

65. The method for placement of an endotracheal tube of any of claims 59-64 and 66, further comprising an actuator.

66. The method for placement of an endotracheal tube of any of claims 59-65, wherein the actuator comprises a tube surrounding a portion of the medial location of the stylet.

67. An intubation tool for assisting in placement of an endotracheal tube, the intubation tool comprising:
a) a stylet having a proximal end, a distal end, and a medial location, the stylet:
i) configured for receiving an endotracheal tube, and
ii) having an articulating distal tip, the articulating distal tip being configured to articulate between more than one shape; and
b) a removable handle secured to a medial location on of the stylet;
wherein the stylet has a substantially uniform outer diameter.

68. The intubation tool of any of claims 67 and 69-71, wherein the minimum non-tip diameter of stylet is within 70 percent of the maximum non-tip diameter of the stylet.

69. The intubation tool of any of claims 67-68 and 70-71, wherein the minimum non-tip diameter of stylet is within 80 percent of the maximum non-tip diameter of the stylet.

70. The intubation tool of any of claims 67-69 and 71, wherein the minimum non-tip diameter of stylet is within 90 percent of the maximum non-tip diameter of the stylet.

71. The intubation tool of any of claims 67-70, wherein the stylet comprises a keyed surface for securing a handle.

72. An intubation tool for assisting in placement of an endotracheal tube, the intubation tool comprising:
a) a stylet having a proximal end, a distal end, and a medial location, the stylet:
i) configured for receiving an endotracheal tube, and
ii) having an articulating distal tip, the articulating distal tip being configured to articulate between more than one shape; and
b) a removable handle secured to a medial location on of the stylet wherein the stylet comprises a flexible cable to articulate the distal tip.

73. The intubation tool for assisting in placement of an endotracheal tube of any of claims 72 and 74-77, wherein the distal end of the stylet is in a neutral position when no articulating force is applied.

74. The intubation tool for assisting in placement of an endotracheal tube of any of claims 72-73 and 75-77, wherein the force to articulate the distal end is less than 2 pounds.

75. The intubation tool for assisting in placement of an endotracheal tube of any of claims 72-74 and 76-77, wherein the force to articulate the distal end is less than 4 pounds.

76. The intubation tool for assisting in placement of an endotracheal tube of any of claims 72-75 and 77, wherein the force to articulate the distal end from 60 to 90 degrees is within 150 percent of the force to articulate the distal end from 0 to 30 degrees.

77. The intubation tool for assisting in placement of an endotracheal tube of any of claims 72-76, wherein the force to articulate the distal end from 80 to 90 degrees is within 150 percent of the force to articulate the distal end from 0 to 10 degrees.

78. An intubation tool for assisting in placement of an endotracheal tube, the intubation tool comprising:
a) a stylet having a proximal end, a distal end, and a medial location, the stylet:
i) configured for receiving an endotracheal tube, and
ii) having an articulating distal tip, the articulating distal tip being configured to articulate between more than one shape, and the articulating distal tip comprises a plurality of free vertebrate; and
b) a removable handle secured to a medial location on of the stylet.

79. The intubation tool of any of claims 78 and 80-86, wherein the vertebrate have a substantially flat peak.

80. The intubation tool of any of claims 78-79 and 81-86, wherein the vertebrate have a substantially flat valley.

81. The intubation tool of any of claims 78-80 and 82-86, wherein the vertebrate interface angle is from 10 to 20 degrees.

82. The intubation tool of any of claims 78-81 and 83-86, wherein handle and stylet provide a substantially clear sight line of at least 90 degrees along a dorsal line of the handle and stylet combination, within 1 cm of the central axis of the stylet.

83. The intubation tool of any of claims 78-82 and 84-86, wherein handle and stylet provide a substantially clear sight-line along the length of the stylet when the stylet is in a non-articulated mode.

84. The intubation tool of any of claims 78-83 and 85-86, wherein the articulating distal tip can be articulated with a force of 4 to 9 Newton.

85. The intubation tool of any of claims 78-84 and 86, wherein the handle of the tool includes a grip extension oriented at 90 degrees to the plane of movement of the distal end of the stylus.

86. The intubation tool of any of claims 78-85, wherein the distal end of the stylet is in a neutral position when no articulating force is applied.

87. An intubation tool for assisting in placement of an endotracheal tube, the intubation tool comprising:
a) a stylet having a proximal end, a distal end, and a medial location, the stylet:
i) configured for receiving an endotracheal tube, and
ii) having an articulating distal tip, the articulating distal tip being configured to articulate between more than one shape; and
b) a removable handle secured to a medial location on of the stylet;
wherein the articulating tip is non-rigid.

88. The intubation tool for assisting in placement of an endotracheal tube of any of claims 87 and 89-92, wherein the force to articulate the distal end from 60 to 90 degrees is within 150 percent of the force to articulate the distal end from 0 to 30 degrees.

89. The intubation tool for assisting in placement of an endotracheal tube of any of claims 87-88 and 90-92, wherein handle and stylet provide a substantially clear sight line of at least 90 degrees along a dorsal line of the handle and stylet combination, within 1 cm of the central axis of the stylet.

90. The intubation tool for assisting in placement of an endotracheal tube of any of claims 87-89 and 91-92, wherein articulating force is applied in-line along the axis of the stylet.

91. The intubation tool for assisting in placement of an endotracheal tube of any of claims 87-90 and 92, wherein handle and stylet provide a substantially clear sight-line along the length of the stylet when the stylet is in a non-articulated mode.

92. The intubation tool for assisting in placement of an endotracheal tube of any of claims 87-91, wherein the handle of the tool includes a grip extension oriented at 90 degrees to the plane of movement of the distal end of the stylus.

93. An intubation tool for assisting in placement of an endotracheal tube, the intubation tool comprising:
a) a stylet having a proximal end, a distal end, and a medial location, the stylet:
i) configured for receiving an endotracheal tube, and
ii) having an articulating distal tip, the articulating distal tip being configured to articulate between more than one shape; and
b) a removable handle secured to a medial location on of the stylet;
wherein the articulating tip transmits the majority of tactile feedback to the vicinity of the removable handle.

94. The intubation tool for assisting in placement of an endotracheal tube of any of claims 93 and 95-99, wherein the force to articulate the distal end is less than 2 pounds.

95. The intubation tool for assisting in placement of an endotracheal tube of any of claims 93-94 and 96-99, wherein the stylet comprises an internal cable connected to the distal end, and wherein pulling on the cable from the medial location results in articulating the distal end of the stylet.

96. The intubation tool for assisting in placement of an endotracheal tube of any of claims 93-95 and 97-99, wherein handle and stylet provide a substantially clear sight-line along the length of the stylet when the stylet is in a non-articulated mode.

97. The intubation tool for assisting in placement of an endotracheal tube of any of claims 93-96 and 98-99, wherein handle and stylet provide a substantially clear sight line of at least 90 degrees along a dorsal line of the handle and stylet combination, within 1 cm of the central axis of the stylet.

98. The intubation tool for assisting in placement of an endotracheal tube of any of claims 93-97 and 99, wherein the force to articulate the distal end from 60 to 90 degrees is within 150 percent of the force to articulate the distal end from 0 to 30 degrees.

99. The intubation tool for assisting in placement of an endotracheal tube of any of claims 93-98, wherein the distal end of the stylet is in a neutral position when no articulating force is applied.

100. An intubation tool for assisting in placement of an endotracheal tube, the intubation tool comprising:
a stylet having a proximal end, a distal end, and a medial location, the stylet:
i) configured for receiving an endotracheal tube;
ii) having an articulating distal tip, the articulating distal tip being configured to articulate between more than one shape; and
iii) a slidable actuator articulating the distal tip, the slidable actuator being engageable around at least 180 degree of the central axis of the stylet.

101. The intubation tool of any of claims 100 and 102, wherein the slidable actuator is engageable around at least 270 of the central axis of the stylet.

102. The intubation tool of any of claims 100-101, wherein the slidable actuator is engageable around at 360 of the central axis of the stylet.

103. An intubation tool for assisting in placement of an endotracheal tube, the intubation tool comprising:
a) a stylet having a proximal end, a distal end, and a medial location, the stylet including a bi-lumen extrusion, the stylet:
i) configured for receiving an endotracheal tube, and
ii) having an articulating distal tip, the articulating distal tip being configured to articulate between more than one shape; and
b) a removable handle secured to a medial location on of the stylet.

104. The intubation tool for assisting in placement of an endotracheal tube of any of claims 103 and 105-109, wherein the force to articulate the distal end from 60 to 90 degrees is within 150 percent of the force to articulate the distal end from 0 to 30 degrees.

105. The intubation tool for assisting in placement of an endotracheal tube of any of claims 103-104 and 106-109, wherein handle and stylet provide a substantially clear sight-line along the length of the stylet when the stylet is in a non-articulated mode.

106. The intubation tool for assisting in placement of an endotracheal tube of any of claims 103-105 and 107-109, wherein the articulating distal tip can be articulated with a force of 4 to 9 Newton.

107. The intubation tool for assisting in placement of an endotracheal tube of any of claims 103-106 and 108-109, wherein the handle of the tool includes a grip extension oriented at 90 degrees to the plane of movement of the distal end of the stylus.

108. The intubation tool for assisting in placement of an endotracheal tube of any of claims 103-107 and 109, wherein handle and stylet provide a substantially clear sight line of at least 90 degrees along a dorsal line of the handle and stylet combination, within 1 cm of the central axis of the stylet.

109. The intubation tool for assisting in placement of an endotracheal tube of any of claims 103-108, wherein the distal end of the stylet is in a neutral position when no articulating force is applied.

110. A method for placement of an endotracheal tube, the method comprising:
a) providing an intubation tool for assisting in placement of an endotracheal tube, the intubation tool comprising:
i) a stylet having a proximal end, distal end, and intermediate medial portion, the stylet configured for receiving an endotracheal tube, and having an articulating distal tip, the articulating distal tip being configured to articulate between more than one shape using one to three fingers; and
ii) a handle secured to a medial position on the stylet;
b) inserting the distal end of the stylet of the intubation tool into the trachea of a person, including articulating the distal end of the stylet during insertion to aid in passage through the larynx.

111. The method for placement of an endotracheal tube of any of claims 110 and 112-114, wherein the force to articulate the distal end from 60 to 90 degrees is within 150 percent of the force to articulate the distal end from 0 to 30 degrees.

112. The method for placement of an endotracheal tube of any of claims 110-111 and 113-114, wherein the stylet comprises an internal cable connected to the distal end, and wherein pulling on the cable from the medial location results in articulating the distal end of the stylet.

113. The method for placement of an endotracheal tube of any of claims 110-112 and 114, wherein the articulating distal tip can be articulated with a force of 4 to 9 Newton.

114. The method for placement of an endotracheal tube of any of claims 110-113, wherein the handle of the tool includes a grip extension oriented at 90 degrees to the plane of movement of the distal end of the stylus.

115. An intubation tool for assisting in placement of an endotracheal tube, the endotracheal intubation tool comprising:
a) a stylet having a medial location and a distal end, the stylet:
i.) configured for receiving an endotracheal tube, and
ii) having an articulating distal tip, the articulating tip end being configured to articulate between more than one shape; and
b) a handle secured to the medial location of the stylet;
wherein the articulating tip comprises vertebrate with a flat portion having a width of W_{f} and the overall vertebrate having a width of Wᵥ, and W_{f} is less than 50 percent of Wᵥ.
